SEQUENCE LISTING (12 sequences)

SEQ ID NO: 1 - protein, 11 aa, Artificial Sequence
  Synthetic peptide
FKKQGSFAKKK

SEQ ID NO: 2 - protein, 11 aa, Artificial Sequence
  Synthetic peptide
FKKQGTFAKKK

SEQ ID NO: 3 - DNA, 8964 bp, Homo sapiens
gtcagtgagcctggggccagcgggcgagccagcggctccggctcccgctcccgctccgcgcagcaccagcccgactctccccggcccccgccgcgccccctcgccgcgacctcggccaccggcccgcgccccgcgcccggggtcgccccgagcccgcacctctcccccgccgcccccgcccacccggccctccgcggccgcagctccccggcggaggcaagaggtggttgggggggaccatggctgacgttttcccgggcaacgactccacggcgtctcaggacgtggccaaccgcttcgcccgcaaaggggcgctgaggcagaagaacgtgcacgaggtgaaggaccacaaattcatcgcgcgcttcttcaagcagcccaccttctgcagccactgcaccgacttcatctgggggtttgggaaacaaggcttccagtgccaagtttgctgttttgtggtccacaagaggtgccatgaatttgttactttttcttgtccgggtgcggataagggacccgacactgatgaccccaggagcaagcacaagttcaaaatccacacttacggaagccccaccttctgcgatcactgtgggtcactgctctatggacttatccatcaagggatgaaatgtgacacctgcgatatgaacgttcacaagcaatgcgtcatcaatgtccccagcctctgcggaatggatcacactgagaagagggggcggatttacctaaaggctgaggttgctgatgaaaagctccatgtcacagtacgagatgcaaaaaatctaatccctatggatccaaacgggctttcagatccttatgtgaagctgaaacttattcctgatcccaagaatgaaagcaagcaaaaaaccaaaaccatccgctccacactaaatccgcagtggaatgagtcctttacattcaaattgaaaccttcagacaaagaccgacgactgtctgtagaaatctgggactgggatcgaacaacaaggaatgacttcatgggatccctttcctttggagtttcggagctgatgaagatgccggccagtggatggtacaagttgcttaaccaagaagaaggtgagtactacaacgtacccattccggaaggggacgaggaaggaaacatggaactcaggcagaaattcgagaaagccaaacttggccctgctggcaacaaagtcatcagtccctctgaagacaggaaacaaccttccaacaaccttgaccgagtgaaactcacggacttcaatttcctcatggtgttgggaaaggggagttttggaaaggtgatgcttgccgacaggaagggcacagaagaactgtatgcaatcaaaatcctgaagaaggatgtggtgattcaggatgatgacgtggagtgcaccatggtagaaaagcgagtcttggccctgcttgacaaacccccgttcttgacgcagctgcactcctgcttccagacagtggatcggctgtacttcgtcatggaatatgtcaacggtggggacctcatgtaccacattcagcaagtaggaaaatttaaggaaccacaagcagtattctatgcggcagagatttccatcggattgttctttcttcataaaagaggaatcatttatagggatctgaagttagataacgtcatgttggattcagaaggacatatcaaaattgctgactttgggatgtgcaaggaacacatgatggatggagtcacgaccaggaccttctgtgggactccagattatatcgccccagagataatcgcttatcagccgtatggaaaatctgtggactggtgggcctatggcgtcctgttgtatgaaatgcttgccgggcagcctccatttgatggtgaagatgaagacgagctatttcagtctatcatggagcacaacgtttcctatccaaaatccttgtccaaggaggctgtttctgtctgcaaaggactgatgaccaaacacccagccaagcggctgggctgtgggcctgagggggagagggacgtgagagagcatgccttcttccggaggatcgactgggaaaaactggagaacagggagatccagccaccattcaagcccaaagtgtgtggcaaaggagcagagaactttgacaagttcttcacacgaggacagcccgtcttaacaccacctgatcagctggttattgctaacatagaccagtctgattttgaagggttctcgtatgtcaacccccagtttgtgcaccccatcttacagagtgcagtatgaaactcaccagcgagaacaaacacctccccagcccccagccctccccgcagtgggaagtgaatccttaaccctaaaattttaaggccacggccttgtgtctgattccatatggaggcctgaaaattgtagggttattagtccaaatgtgatcaactgttcagggtctctctcttacaaccaagaacattatcttagtggaagatggtacgtcatgctcagtgtccagtttaattctgtagaagttacgtctggctctaggttaacccttcctagaaagcaagcagactgttgccccattttgggtacaatttgatatactttccataccctccatctgtggatttttcagcattggaatcccccaaccagagatgttaaagtgagcctgtcccaggaaacatctccacccaagacgtctttggaatccaagaacaggaagccaagagagtgagcagggagggattgggggtgggggaggcctcaaaataccgactgcgtccattctctgcctccatggaaacagcccctagaatctgaaaggccgggataaacctaatcactgttcccaaacattgacaaatcctaacccaaccatggtccagcagttaccagtttaaacaaaaaaacctcagatgagtgttgggtgaatctgtcatctggtaccctccttggttgataactgtcttgatacttttcattctttgtaagaggccaaatcgtctaaggacgttgctgaacaagcgtgtgaaatcatttcagatcaaggataagccagtgtgtacatatgttcattttaatctctgggagattatttttccatccagggtgccatcagtaatcatgccactactcaccagtgttgttcaccaacacccacccccacacacaccaacattttgctgcctaccttgttatccttctcaagaagctgaagtgtacgccctctccccttttgtgcttatttatttaataggctgcagtgtcgcttatgaaagtacgatgtacagtaacttaatggaagtgctgactctagcatcagcctctaccgattgattttcctcccttctctagccctggatgtccacttagggataaaaagaatatggttttggttcccatttctagttcacgttgaatgacaggcctggagctgtagaatcaggaaacccggatgcctaacagctcaaagatgttttgttaatagaaggattttaatacgttttgcaaatgcatcatgcaatgaattttgcatgtttataataaaccttaataacaagtgaatctatattattgatataatcgtatcaagtataaagagagtattataataattttataagacacaattgtgctctatttgtgcaggttcttgtttctaatcctcttttctaattaagttttagctgaatcccttgcttctgtgctttccctccctgcacatgggcactgtatcagatagattactttttaaatgtagataaaatttcaaaaatgaatggctagtttacgtgatagattaggctcttactacatatgtgtgtgtatatatatgtatttgattctacctgcaaacaaatttttattggtgaggactatttttgagctgacactccctcttagtttcttcatgtcacctttcgtcctggttcctccgccactcttcctcttggggacaacaggaagtgtctgattccagtctgcctagtacgttggtacacacgtggcattgccgcagcacctgggctgacctttgtgtgtgcgtgtgtgtgtgtttccttcttcccttcagcctgtgactgttgctgactccaggggtgggagggatggggagactcccctcttgctgtgtgtactggacacgcaggaagcatgctgtcttgctgcctctgcaacgacctgtcgtttgctccagcatgcacaaacttcgtgagaccaacacagccgtgccctgcaggcaccagcacgtgcttttcagaggctgcggactttcttccagccattgtggcattggcctttccagtcttgggaggagcgcgctgctttggtgagacacccccatgcaaggtcctcagagtagccgggttctaccacaaacagaaacagaatgaaagtagctgtcagtccttgtagagagccgctctgtttcctcccagaagcatctcccagctaagctcgcattatttttctcctctggctgtttgcctgaagttcacagaacacacaaccatgaaaggctttttgaggtgagaggcccaggtggtcctggcaaccctgagtagaaggagagacggggtagggaacgggcccggccagaaaagaaccatttcttctgccatcttttatgcaccatagacatcgagactccagggggtcctggctcccctgtccctgcagccctgcaggtcagtgcatgatctgggttcgtgtcctgaccaggtgctcctcctttgatccgaggggaaagggactggtttatagaaagagcctaggagacaaaagggccagtccccctgcccagaatggagcagcagcaggacagacccccacgaggccccccagagaggaggaagatcccacggaggaacacatgaggttagggacccttgttcagcaccccaaacagcctgcctgtttaaagcaggcagcaggcttaggccttccctgcaaccccaacacccacaagtttgtttctctaggaaacacattcactgtctcagctggctgttactctctcagaccatatggcaaagttttccaagaaaatgccccgacaggggtgcccagcacactgcctgagggacaacagacatcagaacaaacccccagagagaaacagtcaaaatcagggcccggtgcagtgttgtcatgtggaacctgctttatccattgctgagtgttgaatgtgggtaatggttagggctttccagatctcagcagccaaagacagttattgttggaagactgtcatgtagataaccatgagcaatggctcgcctcagaatcagttcataaaattctatggtactggccccttcgtgggtattgtgtgaaatgagatggtggcgaggggtgcgctgtggaactgccgcagccacgcaggaggtccctgggggatgctttgggaagtccttgcccctgagcactgcctgattgccagggcctgtggaggtctaggccgcctggcagaatctagcaccgtccgaatccccgcaggacccatggagctatgaccacaccaggccattcaaatggctctgcattatcttcccttggaaggtggccactcctcggtggcagggcctttccctgaggctgcaggccgtgggctggcagcccgtctcttggcatttcaattgaaggtcaccaggtgctgggtttgaaaggaagtcactggagtgctgccaggggccgccctccaaggttaatgagaggcccacatccaggcaagaactaattcaaaaggcagatcagaaaccacaggagtcaaaattattgctccggcagtgcttcccttcctttcatccactggcctcgtgtggtccatgcagggccactgtctgccctttctgatgccacgtattaggctttcttactcagaattttgatagaaaaccatggggccaagagctctggaagcctggccggaaagaccaaggttcatgcagcccaacaaatgattgttgagcacctctcggagccaaagtccttaggcgagtgtggtgacttcctggaaggaggatgcagacttccagagagcccccccaacggacgtgctgagaagggagagggaggcgggggctgtagtcaggaaggagccagagaagaacagggtttgggtgcatccagaaatatgcctgcagtaggagggagaggaaggggtgccaccgtcaacggcttcccatcggaggtggttggtgcagatggaagtttctgtctgctggccctcaagagagtgttttgccagggacacagtctgttcctcctcagaaaacaccccccaaatgctaacaacatccccaccagctgctagaagcccctttcccctccccaccttgaagtagctcatagttctctgggcagagccagaccatccagtgtaccccagaggccagtaggttcctgcccattttcctctctggcttcctgccaagaattatggcagctgaggatgaatggagaagtaaaaacaactaacaccgcacaactaacaactaacaccgcagttcccacctgggttccacttagcaggagacatttcggagggttttttttgtttttgttcctgttttttttttttttgctggaatttgttttctcagtactgaaaagagaaaaagtgacaatcttgtatttttaaaagcctcggaaaggtgataccatctgacagtcattttctcacgttggtcttctaaagtcacctatttcttgtgtgtgcacatcacaccatttcctgtttctttataacccgacaagggtaggagtgcctgtttcccctgctgggcacaccagacaatcgtaatcacaaaacagacactgagccaggggcccaaagggtgtgatcatgagagttaccgggacagcagtaggcatgacagtcaccaggaaggacaagggtgctctgttgttagtggccacacaccaatttgacaaggagtgttgcgaaatttttatttatttatttatttattttgagatggagtttcactcttgttgcccaggctggagtgcggtggtacaatctcggctcactgcaacctccacctcccaggttcaagcgattctcctgcctcagcctcccaagtacctgggactacaggtgcgtgccaccacacccagctaaattttgtgtttttagtagagatggggtttcaccatgttggccaggatggtcttgaacccctgacctcatgatctgcctgcctcggcctcccaaagtgctgggattacaggcatgagccaccacgcccagccaaaatatttttttaaagtcattttccttaagctgcttgggctacatgtgaaatacactggacggtcaacattcctgtctcctcccatttgggctgatgcagcagatccagggaatgttacctgtttctgctgctagaagatccaggaaattgggaaggttacctgacgcacacatggatgaaggccatcatctagaaatggggtcaaccacaattgtgttaattccgtagtgtcagggattcttcgggaaggtcaacagtatgaaggattctgacccctgtgcctcccatttatgtgatcaggtgacagttaataaccgtggaggtcacactcagccatccaacagccttacagtgaccctacacaaaagcccccaaattccaaagactttttcttaacctaaaggaagaaattatttgttaattccagtagagcaactgaatatactgggctatttgtacttttttatagagaactttaataataattctttaaaaatgagtttttagaacaaagcaactgacgatttcctaagattccaatgccctggagcttgtaggaggacttagcctgggtcagctggagcacccccgacctgatctcccactgccagattttcccatgctcctagggtatggagtccacgtgggaatgactgcaagttcaggtggaacttggccgactgatgctctgcgagtttttaatagacactggggacaactgcttaaggtttagaaacttccaaaccacaggaaagacatttttagtgtcccccatccagaggcagccctggaataggattcccaggggtttctgggacccctttccttgctccgtgaggctctgtggccatcttttggcaggaggaggatgcttccttggctctgtgcccagacccgcctggtccccaggtctctcaccttgggtgaagattcagagatgccctgtaaggattttgcccactgggcaactcagaaatacttcgatctcccaagatataagaggcagcagcaaacgtgcctattgacgtctgtttcatagttaccacttacgcgagtagacagaactcggcttttcagaaaataggtgtcaagtccactttataagaacctttttttctaaaataagataaaaggtggctttgcattttctgattaaacgactgtgtctttgtcacctctgcttaactttaggagtatccattcctgtgattgtagacttttgttgatattcttcctggaagaatatcattcttttcttgaagggttggtttactagaatattcaaaatcaatcatgaaggcagttactattttgagtctaaaggttttctaaaaattaacctcacatcccttctgttagggtctttcagaatatcttttataaacagaagcatttgaagtcattgcttttgctacatgatttgtgtgtgtgaaggacataccacgtttaaatcattaattgaaaaacatcatataagccccaactttgtttggaggaagagacggaggttgaggtttttccttctgtataagcacctactgacaaaatgtagaggccattcaaccgtcaaacaccatttggttatatcgcagaggagacggatgtgtaaattactgcattgctttttttttcagtttgtataacctctaatctccgtttgcatgatacgctttgttagaaacattaattgtagtttggaagcaagtgtgtatgaataaagataatgatcattc

SEQ ID NO: 4 - protein, 672 aa, Homo sapiens
MADVFPGNDSTASQDVANRFARKGALRQKNVHEVKDHKFIARFFKQPTFCSHCTDFIWGFGKQGFQCQVCCFVVHKRCHEFVTFSCPGADKGPDTDDPRSKHKFKIHTYGSPTFCDHCGSLLYGLIHQGMKCDTCDMNVHKQCVINVPSLCGMDHTEKRGRIYLKAEVADEKLHVTVRDAKNLIPMDPNGLSDPYVKLKLIPDPKNESKQKTKTIRSTLNPQWNESFTFKLKPSDKDRRLSVEIWDWDRTTRNDFMGSLSFGVSELMKMPASGWYKLLNQEEGEYYNVPIPEGDEEGNMELRQKFEKAKLGPAGNKVISPSEDRKQPSNNLDRVKLTDFNFLMVLGKGSFGKVMLADRKGTEELYAIKILKKDVVIQDDDVECTMVEKRVLALLDKPPFLTQLHSCFQTVDRLYFVMEYVNGGDLMYHIQQVGKFKEPQAVFYAAEISIGLFFLHKRGIIYRDLKLDNVMLDSEGHIKIADFGMCKEHMMDGVTTRTFCGTPDYIAPEIIAYQPYGKSVDWWAYGVLLYEMLAGQPPFDGEDEDELFQSIMEHNVSYPKSLSKEAVSVCKGLMTKHPAKRLGCGPEGERDVREHAFFRRIDWEKLENREIQPPFKPKVCGKGAENFDKFFTRGQPVLTPPDQLVIANIDQSDFEGFSYVNPQFVHPILQSAV

SEQ ID NO: 5 - DNA, 2245 bp, Homo sapiens
ggagcaagaggtggttggggggggaccatggctgacgttttcccgggcaacgactccacggcgtctcaggacgtggccaaccgcttcgcccgcaaaggggcgctgaggcagaagaacgtgcacgaggtgaaggaccacaaattcatcgcgcgcttcttcaagcagcccaccttctgcagccactgcaccgacttcatctgggggtttgggaaacaaggcttccagtgccaagtttgctgttttgtggtccacaagaggtgccatgaatttgttactttttcttgtccgggtgcggataagggacccgacactgatgaccccaggagcaagcacaagttcaaaatccacacttacggaagccccaccttctgcgatcactgtgggtcactgctctatggacttatccatcaagggatgaaatgtgacacctgcgatatgaacgttcacaagcaatgcgtcatcaatgtccccagcctctgcggaatggatcacactgagaagagggggcggatttacctaaaggctgaggttgctgatgaaaagctccatgtcacagtacgagatgcaaaaaatctaatccctatggatccaaacgggctttcagatccttatgtgaagctgaaacttattcctgatcccaagaatgaaagcaagcaaaaaaccaaaaccatccgctccacactaaatccgcagtggaatgagtcctttacattcaaattgaaaccttcagacaaagaccgacgactgtctgtagaaatctgggactgggatcgaacaacaaggaatgacttcatgggatccctttcctttggagtttcggagctgatgaagatgccggccagtggatggtacaagttgcttaaccaagaagaaggtgagtactacaacgtacccattccggaaggggacgaggaaggaaacatggaactcaggcagaaattcgagaaagccaaacttggccctgctggcaacaaagtcatcagtccctctgaagacaggaaacaaccttccaacaaccttgaccgagtgaaactcacggacttcaatttcctcatggtgttgggaaaggggagttttggaaaggtgatgcttgccgacaggaagggcacagaagaactgtatgcaatcaaaatcctgaagaaggatgtggtgattcaggatgatgacgtggagtgcaccatggtagaaaagcgagtcttggccctgcttgacaaacccccgttcttgacgcagctgcactcctgcttccagacagtggatcggctgtacttcgtcatggaatatgtcaacggtggggacctcatgtaccacattcagcaagtaggaaaatttaaggaaccacaagcagtattctatgcggcagagatttccatcggattgttctttcttcataaaagaggaatcatttatagggatctgaagttagataacgtcatgttggattcagaaggacatatcaaaattgctgactttgggatgtgcaaggaacacatgatggatggagtcacgaccaggaccttctgtgggactccagattatatcgccccagagataatcgcttatcagccgtatggaaaatctgtggactggtgggcctatggcgtcctgttgtatgaaatgcttgccgggcagcctccatttgatggtgaagatgaagacgagctatttcagtctatcatggagcacaacgtttcctatccaaaatccttgtccaaggaggctgtttctatctgcaaaggactgatgaccaaacacccagccaagcggctgggctgtgggcctgagggggagagggacgtgagagagcatgccttcttccggaggatcgactgggaaaaactggagaacagggagatccagccaccattcaagcccaaagtgtgtggcaaaggagcagagaactttgacaagttcttcacacgaggacagcccgtcttaacaccacctgatcagctggttattgctaacatagaccagtctgattttgaagggttctcgtatgtcaacccccagtttgtgcaccccatcttacagagtgcagtatgaaactcaccagcgagaacaaacacctccccagcccccagccctccccgcagtggaagtgaatccttaaccctaaaattttaaggccacggcttgtgtctgattccatatggaggcctgaaaattgtagggttattagtccaaatgtgatcaactgttcagggtctctctcttacaaccaagaacattatcttagtggaag

SEQ ID NO: 6 - protein, 672 aa, Homo sapiens
MADVFPGNDSTASQDVANRFARKGALRQKNVHEVKDHKFIARFFKQPTFCSHCTDFIWGFGKQGFQCQVCCFVVHKRCHEFVTFSCPGADKGPDTDDPRSKHKFKIHTYGSPTFCDHCGSLLYGLIHQGMKCDTCDMNVHKQCVINVPSLCGMDHTEKRGRIYLKAEVADEKLHVTVRDAKNLIPMDPNGLSDPYVKLKLIPDPKNESKQKTKTIRSTLNPQWNESFTFKLKPSDKDRRLSVEIWDWDRTTRNDFMGSLSFGVSELMKMPASGWYKLLNQEEGEYYNVPIPEGDEEGNMELRQKFEKAKLGPAGNKVISPSEDRKQPSNNLDRVKLTDFNFLMVLGKGSFGKVMLADRKGTEELYAIKILKKDVVIQDDDVECTMVEKRVLALLDKPPFLTQLHSCFQTVDRLYFVMEYVNGGDLMYHIQQVGKFKEPQAVFYAAEISIGLFFLHKRGIIYRDLKLDNVMLDSEGHIKIADFGMCKEHMMDGVTTRTFCGTPDYIAPEIIAYQPYGKSVDWWAYGVLLYEMLAGQPPFDGEDEDELFQSIMEHNVSYPKSLSKEAVSICKGLMTKHPAKRLGCGPEGERDVREHAFFRRIDWEKLENREIQPPFKPKVCGKGAENFDKFFTRGQPVLTPPDQLVIANIDQSDFEGFSYVNPQFVHPILQSAV

SEQ ID NO: 7 - DNA, 8385 bp, Mus musculus
agtgagcgtggggccagccagcgagccagagagccggagagagccagagagagcggctcggctcccagctccgagtagcgcagcgcccgcccggctctcccgggccaccgccgccaccaccgcacctcatcagcaccgccacctcggccgccgcccccgcccaccccggccctccccggctgctgctccccggcggaggcaagaggtggttggggggaaccatggctgacgtttacccggccaacgactccacggcgtctcaggacgtggccaaccgcttcgcccgcaaaggggcgctgaggcagaagaacgtgcatgaggtgaaagaccacaaattcatcgcccgcttcttcaagcaacccaccttctgcagccactgcaccgacttcatctgggggtttgggaaacaaggcttccagtgccaagtttgctgttttgtggttcataagaggtgccatgagttcgttacgttctcttgtccgggtgcggataagggacctgacactgacgaccccaggagcaagcacaagttcaaaatccacacatacggaagccctaccttctgtgatcactgtgggtccctgctctatggacttatccaccaagggatgaaatgtgacacctgcgacatgaatgttcacaagcagtgtgtgatcaatgtccctagcctctgcggaatggatcacacagagaagagggggcggatttatctgaaggctgaggtcactgatgaaaagctccacgtcacggtacgagatgcaaaaaatctaatccctatggatccaaatgggctttcggatccttatgtgaagctgaaacttatccctgaccccaagaatgagagcaaacagaaaaccaaaaccatccgctccacactgaatcctcagtggaatgagtccttcacgttcaaattaaaaccttcagacaaagaccggcgactgtctgtagaaatctgggactgggatcggacgactcggaatgacttcatgggatccctttcctttggtgtctcagagctaatgaagatgccggccagtggatggtataaactgctcaaccaagaagagggcgaatactacaatgtgcccattccagaaggagatgaagaaggcaacatggaactcaggcagaagtttgagaaagccaagctaggccctgctggtaacaaagtcatcagcccttcagaagacagaaagcaaccatccaacaacctggacagagtgaaactcacagacttcaacttcctcatggtgctggggaaggggagttttgggaaggtgatgcttgctgacaggaagggaacggaggaactgtacgccatcaagatcctgaagaaggacgtggtgatccaggacgacgacgtggagtgcaccatggtggagaagcgcgtgctggccctgctggacaagccgccatttctgacacagctgcactcctgcttccagacagtggaccggctgtacttcgtcatggaatacgtcaacggcggggacctcatgtaccacattcagcaagtcgggaaatttaaggagccacaagcagtattctacgcagccgagatctccatcggactgttcttccttcataaaagagggatcatttacagggatctgaagctggacaatgtcatgctggactcagaagggcacatcaaaatcgccgactttgggatgtgcaaggaacacatgatggatggagtcacgaccaggaccttctgtgggactccggactacattgccccagagataatcgcttaccagccgtacgggaagtctgtagattggtgggcgtacggtgtgctgctgtacgagatgctagccgggcagcctccgtttgatggtgaagatgaagatgaactgtttcagtctataatggagcacaacgtgtcctaccccaaatccttgtccaaggaagccgtctctatctgcaaaggacttatgaccaaacaccctgccaagcggctgggctgcgggcccgagggagagagggatgtcagagagcatgccttcttcaggagaatcgactgggagaaactggagaacagggagatccaaccaccattcaagcccaaagtgtgtggcaaaggagcagaaaactttgacaagttcttcacgcgaggacagcctgtcttaacaccaccagatcagctggtcattgctaacatagaccaatctgattttgaagggttctcgtatgtcaacccccagtttgtgcacccaatcttgcaaagtgcagtatgaaactcaaaaatgaaagatcaaatgcctccccagcccccagcatccccagcagtgggaaatgatccttaaccataaaaatttaaggctatggccttgtattttgttccacacagaggcctggaaattctagggacattagtcaatacatgatcaacttttttcctcaccccccaaccaaaagcattatcttagtggatgatgacataaagtacagagtatagtttaattatgtagaagtcatgtcagctacaagttgattcttcctaaaaaaaatcaaagcgacttggaccctattttttttgtacgatttgatatattctccatactttccatttcttggattttcactatcagaatcgaccagagatagtaaagtgaactcaccctaactcaaggaaactcaaggaaacacttccacccaagatgtctttggaacaaaagaacaggaagtccaaagagaaagcaaagagaggttgggggctcatatattcaagaccacatagcttctattctccttttcaatggaaacagtccctagactcagagagggcaggatgaacctgatcactgttcccagtcatcatagcacaaccatagtgcacagtttaagaaggaaagaaaacttcagacaaatattcactgaatctaccatctgtaatcccctgcttggttgataacaatctctataactcattctttttaagaggccaaatcatctaaggactttgctaaacaagcatgtgaaatcatttcagatcaaggataaaccatgtgtacgttcattctaatctctgggagatgactcttcaatccagggtgccatcagtaatcatgccactgttcacgagtgttgttagccaaccccacacacacaatattttgctacctttgtaggtacccttcctaagaagctgaaatgtatgaccccatccccccccctttttttactatttatttaataagctgcagtgtcgtttatggaagtacaatgtatagtaatttagtcaaagtattgactagcctcagtccctctagattgattttcctcctttctctcgccccacatccacttagagatgaaaaagagcatagtttgggttcccattctcagttcctattgaacggcacatgtggaactgtaggatcaagatactttatgctgatatcaactcaaagatgttgtcttgctagaaggattttaatatgttttgcgagtgcatcatgcaatggattttgcatgtttataataaaccttaataacgagttaatctatattattgatataattgtatcaactataaagagtattatgataatttttataagacacaattgtgctatatttgtgacgctcatgtttccagtctgctttaagattaagtgttagctttattcattgctgctgggctctcacccgctactccacacactggcactctatcagatatattaattttttaatgtagatgtaattttaaaaatgaatggctactttacacgtttaactagggttttactatatatgtgtacatatgtatatatatatatatatatatttgattctacctacaaaccccggttttgtttgtggggattattttttgagataagactctgttaacttcttcatgttatcctctgttcaagttcctttatcaccccttctcttggggacagctgaatatatttaattccagtattcctgatatgttcatatatatgtggcattgttacagcacctaggctgaattgtgtgtgtgtgtgtgtctgtctgtctgtctgtctgtctgtctggtacttttcagcctatgtctttggggtctctagggatgaaggggtgagagaactcctttctgtgtgtgcatatggacacccaaaaatatgtagtctgtctttctgccccagaataactcactgtgtacttcagaacacacagaatccttgagacaaactttatctccatgcctgtgtagagccatctctgaagctgttgccaacagactctcttcagaggcacatactgtcttaaagcggttcctagaacattccggtctttgaagagcatgcctttgtccaaatgcctcctaatacaaggtgtttagaagaggcaggcttctgcagcataaaggataagtgaaaaaggagttcctaccagtccttagagaaggcacctctatcgaaatcatctcacagctaggctctcctctttgttattctccacctatctgcttgacagtcacataccctgcaacccaccaagagtctttgagatacggagcataggctgttctgaaaatccctaatcagaaggagatatggagtcgagagtgggccaaaccagaaaagagcagtttcttctgcaatctttcatgaattctggacttcagacctcaaaaagtggggaagggtgtcatatatggatctcagaagtcaagttcaccatcttggtaacctagccttatcaaaaacactgtactcagtccaactgtaaaggaactggcttatcaatgagccttggaagtgaaagcattgctcttcccaggtgggacagcaacaggaagctgaacctctcaaagtgggggaaaccacagagaaggagcacagggttcgtagacaatccaccctgaggtgccaagctgcctgactctcatgccagtagcaggcttgggttcttccttaaaccgccagcatccagctccgagtgattcaacttctctagaaaatgtgtgcggttccttggatggcttgtacgcttagagaatacccaccaacattctcacactggaggagggaaataacagagccacagaccagcaattgcctaaggaacagaacactggagaacaaacttaggagagatgataaaaatttaaggacgtgttcatgatgctgcctattctatacactgctgagggttaaattcagatgaaggctagtactttaaagatctggacagcagagacaacttttgctacaaaactaactcacctggataaccactagcagctgttaggcaggatcaattgatactattctacatccttgtcccacttcacagaccttgtatgaaacagggctacagggaaacatctgccatttgaactgaagcaaccaagcaggaggccagaaccaggagggtgccttgaagggtttaatccccaggggcaggggtgggaactatttagaatgtacctgctctcaggctgtgcctgcattggcagggtctgtggaggagaacctagcacaatccaagtctccacagggacccatcaggccgtgacccagaaaatccattgaaataattttgtattatattcccctggaaggtggccatttcccaggagcagctcttccctgttccaccacaggccggtgggctgctagctctgccttcagtgtggcatctaaagttacctggtgcttgggttgaatggaaagagctggagtatgctgccaggtgcagccccaggagttaatgggaggcttaccccaggcagcagctaattcagaaggcagaagagaagccccaggagctaaactgattgcccttcaccctcctacctctgtggcctagtttgagctagtagccttttctttaatttagtcttttttattagtaatttgagaaactcaggaggcccagagttttccggaagcttggtctgagatggtcacgtgaaagattaccagaggccagtattaaccctcaaatgttgctgctcacaagctccagttcttagcaaataaatcatggtggcttgccaaccccaaaagagccactcatcgatgtatcacaaagaggaggaagcagaagtcaacaccattaaagagcagggtttgagtgcacccaggaagacatggaaggtggtggaaaggttgtgttaatctgcatgggctttccactgggggtcattgtgcaaatggaagttactgttcatcagccttcaaaggtgtggctttttgccagatacaaccttttgttcgttctcagactctcccaactgtaacaagtcctttgcccagagccgagccagccagcagaccctagaagcacttagttccatctttatccctctgataattatggctgctgagactaggtccagaagtcacgaaaacaaccaacaacgacctgccagcttctgctcagcagagaaaaataggaagggtttgtgcagggctttattttctcaaacgcctccacaaggcaacaccacctgacagtcattttctcaccttggtcttatgaaatcatctatttcttgtgtgtacatgtcacgctgtttcctgtttgtttacaaccctagaagggtagggttacctgtccccatgccacggctgagcacatcagtcactctcaggagatgcgtgctgagccagggacctgaaaggatgctgaccatggggggttggggggacaatagcaaggaaggagacaggtcctctggttagcagtgggtccacactaacatgacagagtgttaggaaactggttttaaaattgttttccatgaactgctgttaacaaaatgtgctggacagtcaacacccctgccacatctcactgggccagcgtagcaaatctagaagaagcagatactttctgctgtcagaataatccagaaaatcaggaaagggtgcttgaggcatgcacagacacagtgttctagaaacggtgtctaatgctgtcccagggatgatacagcaatggttaaaaagtgtggttggctcagcattcgtgtggtcaggtgtcagtcaatgaacagaagacacaccaggttgttaaacagccttactgtgaccctcctcagaagcccacaagttccaaagactttttttaacctaaaacaagaaattaacttattaactccaagagagtacctgggtatcctgggctatttgtacttttttatagaggactttaataactcaattcttttcaagtgagtttttaaaataataaaaatttcataaatttccaattccatgcaacttataggaggatttcacccaggttggatagagcacccacgatctgcattcccacagcgggattttcccgtgctagtgaggtctggagtccacattggattggctggcatttcctagcagacactaaagatagctgtgcaaggtttagctatcgctcaactgtggggatgacggctttatagctccccttcctgatacagcccccggaaaggattcttggggatctacaggaccccactcttcactgtaagagtcagagcaggaagggggtgcttcttcaactctggctcaagtcccctctggtctctcagtctctcgcatttgattaaaatggagagatgccctctaaggattctgcccactgggcaattcaagcctcctaccccgagaagagacagcgctgagtgtgcctgggcatctgcgtccatgcccatggatgtgagtagacagaaccctacttttgagaaaacgcctatccagcccactttgtaagaaccccctttttctaaactatgacaaaaggcggctttgcactttctgatcaaacaacactgtcttttgtctcctgcttaacttttaagactattctgtgaccgacgtttgtagatttttttttttgttaatattttgcctggaaaaatgtgactctctccttgggaggtttagtagactaataaaaattatacatgaaggtggttgctattttgaggctaaaggtttttctaagaatcgccctcgtatcctgaccgtgaaggcttttcaggataccttttatagacgatcagatgcattcgagctgtgtttcctacatgatttctgttttgttttatgaaggacacgccatgtttaaatctctcattggaaaaaaaaaaaaaaaacaccatgcaaaaccccaaattggtttggaggaaggagtgaaggtggaggctgttgcccgaatgggcctttactgatgagccaggggctgatccactgctcgtttggttactgggcaggtgaggtagacatgtaaattactgcctagctttcttggatttgtgtaacctctactctccgtttgcatgatccgctctgttagatgcattcattgtagttcggaagcaagcgtgtatgaataaagatactgatcactgctgaaaaaaaaaaaaaaaaa

SEQ ID NO: 8 - protein, 672 aa, Mus musculus
MADVYPANDSTASQDVANRFARKGALRQKNVHEVKDHKFIARFFKQPTFCSHCTDFIWGFGKQGFQCQVCCFVVHKRCHEFVTFSCPGADKGPDTDDPRSKHKFKIHTYGSPTFCDHCGSLLYGLIHQGMKCDTCDMNVHKQCVINVPSLCGMDHTEKRGRIYLKAEVTDEKLHVTVRDAKNLIPMDPNGLSDPYVKLKLIPDPKNESKQKTKTIRSTLNPQWNESFTFKLKPSDKDRRLSVEIWDWDRTTRNDFMGSLSFGVSELMKMPASGWYKLLNQEEGEYYNVPIPEGDEEGNMELRQKFEKAKLGPAGNKVISPSEDRKQPSNNLDRVKLTDFNFLMVLGKGSFGKVMLADRKGTEELYAIKILKKDVVIQDDDVECTMVEKRVLALLDKPPFLTQLHSCFQTVDRLYFVMEYVNGGDLMYHIQQVGKFKEPQAVFYAAEISIGLFFLHKRGIIYRDLKLDNVMLDSEGHIKIADFGMCKEHMMDGVTTRTFCGTPDYIAPEIIAYQPYGKSVDWWAYGVLLYEMLAGQPPFDGEDEDELFQSIMEHNVSYPKSLSKEAVSICKGLMTKHPAKRLGCGPEGERDVREHAFFRRIDWEKLENREIQPPFKPKVCGKGAENFDKFFTRGQPVLTPPDQLVIANIDQSDFEGFSYVNPQFVHPILQSAV

SEQ ID NO: 9 - DNA, 3524 bp, Mus musculus
gccgcgagtaggaagcgcgagcgccgggcgcgggctgtcagtgagcgtggggccagccagcgagccagagagccggagagagccagagagagcggctcggctcccagctccgagtagcgcagccgcccggctctccccggcaccgccgccaccaccgcacctcatcagcaccgccacctcgccgccgcccccgcccaccccggccctccccggctgctgctccccggcggaggcaagaggtggttggggggaaccatggctgacgtttacccggccaacgactccacggcgtctcaggacgtggccaaccgcttcgcccgcaaaggggcgctgaggcagaagaacgtgcatgaggtgaaagaccacaaattcatcgcccgcttcttcaagcaacccaccttctgcagccactgcaccgacttcatctgggggtttgggaaacaaggcttccagtgccaagtttgctgttttgtggttcataagaggtgccatgagttcgttacgttctcttgtccgggtgcggataagggacctgacactgacgaccccaggagcaagcacaagttcaaaatccacacatacggaagccctaccttctgtgatcactgtgggtccctgctctatggacttatccaccaagggatgaaatgtgacacctgcgacatgaatgttcacaaccagtgtgtgatcaatgaccctagcctctgcggaatggatcacacagagaagagggggcggatttatctgaaggctgaggtcactgatgaaaagctccacgtcacggtacgagatgcaaaaaatctaatccctatggatccaaatgggctttcggatccttatgtgaagctgaaactaatccctgaccccaagaatgagagcaaacagaaaaccaaaaccatccgctccaacctgaatcctcagtggaatgagtccttcacgttcaaattaaaaccttcagacaaagaccggcgactgtctgtagaaatctgggactgggatcggacgactcggaatgacttcatgggatccctttcctttggtgtctcagagctaatgaagatgccggccagtggatggtataaagctcacaaccaagaagagggcgaatattacaacgtgcccattccagaaggagatgaagaaggcaacatggaactcaggcagaagtttgagaaagccaagctaggtcctgttggtaacaaagtcatcagcccttcagaagacagaaagcaaccatccaacaacctggacagagtgaaactcacagacttcaacttcctcatggtgctggggaaggggagttttgggaaggtgatgcttgctgacaggaagggaacggaggaactgtacgccatcaagatcctgaagaaggacgtggtgatccaggacgacgacgtggagtgcaccatggtggagaagcgcgtgctggccctgctggacaagccgccatttctgacacagctgcactcctgcttccagacagtggaccggctgtacttcgtcatggaatacgtcaacggcggggatcttatgtaccacattcagcaagtcgggaaatttaaggagccacaagcagtattctacgcagccgagatctccatcggactgttcttccttcataaaagagggatcatttacagggatctgaagctgaacaatgtcatgctgaactcagaagggcacatcaaaatcgccgacttcgggatgtgcaaggaacacatgatggatggagtcacgaccaggaccttctgcggaactccggactacattgccccagagataatcgcttaccagccgtacgggaagtctgtagattggtgggcgtacggtgtgctgctgtacgagatgctagccgggcagcctccgtttgatggtgaagatgaagatgaactgtttcagtctataatggagcacaacgtgtcctaccccaaatccttgtccaaggaagccgtctccatctgcaaaggacttatgaccaaacagcctgccaagcgactgggctgcgggcccgagggagagagggatgtcagagagcatgccttcttcaggaggatcgactgggagaaactggagaacagggagatccaaccaccattcaagcccaaagtgtgtggcaaaggagcagaaaactttgacaagttcttcacgcgaggacagcctgtcttaacaccaccagatcagctggtcattgctaacatagaccaatctgattttgaagggttctcgtatgtcaacccccagtttgtgcacccaatcttgcaaagtgcagtatgaactcaaaaatgaaagatcaaatgcctccccagcccccgcatccccatcagtgggaagtgatccttaaccataaaaatttaaggctatggctttgtattttgttccacacagaggcctggaaattttagggacattagtaatacattatcaacttttttcctcaccccccaaccaaaagcattatcttagtggatgatgacataaagtacagagtatagtttaattatgtagaagtcatgtccggctacaagttgattcttcctaaaaatcaaagagacttggaccctattttttggtacgatttgatatattctccatactttccatttcttggattttcagtatcagtatcaaccagagatagtaaagtgacccaccctaactcaaggaacacttccacccaagatgtctttggaacaaagacaggaagtccaaagagaaagcaaagagaggttgggggctcatatattcaagaccacatagcttctattcttcgtttcaatggaaacatccctagactcagagagggcaggatgaacctgatcactgttcccagtcatcatagcacaaccatagtgcacagtttaagaatgaaagaaaacttcagacaaatattcactgaatctaccatctgtaatcccctgcttagttgataacaatctctataactcattctttttaagaggccaaatcatctaagggctttgctaaacaagcatatgaaatcatttcagatcaaggataaaccatgtgtacgttcattctaatctctgggagatgactcttcaatccagggtgccatcagtaatcatgccactgttcacgagtgttgttagccaaccccacacacacaatattttgctacctttgtaggtacccttcctaagaagatgaaatgtatgaccccatccccccctttttttttactatttatttaataagctgcagtgtcgtttatggaagtacaatgtatagtaatttagtcaaagtattgactagcctcagtccctctagattgattttcctcctttctctcgccccacatccacttagagatgaaaaagagcatagtttgggttcccattctcagttcctattgaacggcacatgtggaactgtaggatcaagatactttatgctgacatcaactctttgatgttgtcttgctagaaggattttaatatgttttgcgagtgcatcatgcaatggattttgcatgtttataataaaccttaataacgagttaagtg

SEQ ID NO: 10 - protein, 672 aa, Mus musculus
MADVYPANDSTASQDVANRFARKGALRQKNVHEVKDHKFIARFFKQPTFCSHCTDFIWGFGKQGFQCQVCCFVVHKRCHEFVTFSCPGADKGPDTDDPRSKHKFKIHTYGSPTFCDHCGSLLYGLIHQGMKCDTCDMNVHNQCVINDPSLCGMDHTEKRGRIYLKAEVTDEKLHVTVRDAKNLIPMDPNGLSDPYVKLKLIPDPKNESKQKTKTIRSNLNPQWNESFTFKLKPSDKDRRLSVEIWDWDRTTRNDFMGSLSFGVSELMKMPASGWYKAHNQEEGEYYNVPIPEGDEEGNMELRQKFEKAKLGPVGNKVISPSEDRKQPSNNLDRVKLTDFNFLMVLGKGSFGKVMLADRKGTEELYAIKILKKDVVIQDDDVECTMVEKRVLALLDKPPFLTQLHSCFQTVDRLYFVMEYVNGGDLMYHIQQVGKFKEPQAVFYAAEISIGLFFLHKRGIIYRDLKLNNVMLNSEGHIKIADFGMCKEHMMDGVTTRTFCGTPDYIAPEIIAYQPYGKSVDWWAYGVLLYEMLAGQPPFDGEDEDELFQSIMEHNVSYPKSLSKEAVSICKGLMTKQPAKRLGCGPEGERDVREHAFFRRIDWEKLENREIQPPFKPKVCGKGAENFDKFFTRGQPVLTPPDQLVIANIDQSDFEGFSYVNPQFVHPILQSAV

SEQ ID NO: 11 - DNA, 3702 bp, Rattus norvegicus
gccgcgagtaggaagcgcgagcgccaggcgccgggctgtcagtgagcgtggggccagccagagagcgagagagccggagagagccagagagagccagagagagcggctcagctcccagctccaagcagcgcagcgcccgcccggctctccccggccaccgccgccaccaccgcacctcagcaccgccacctcggccgccgcccccgcccaccccggccctccccggctgctgctccccggcggaggcaagaggtggttgggggggaccatggctgacgtttacccggccaacgactccacggcgtctcaggacgtggccaaccgcttcgcccgcaaaggggcgctgaggcagaagaacgtgcatgaggtgaaagaccacaaattcatcgcccgcttcttcaagcaacccaccttctgcagccactgcaccgacttcatctgggggtttggaaaacaaggcttccagtgccaagtttgctgttttgtggttcacaagaggtgccatgagtttgttactttctcttgtccgggtgcggataagggacctgacactgatgaccccagaagcaagcacaagttcaaaatccacacctatggaagccctaccttctgtgatcactgtgggtccctgctctacggacttatccaccaagggatgaaatgcgacacctgcgacatgaatgttcacaagcagtgcgtgatcaatgtccccagcctctgcggaatggatcacacagagaagagggggcggatttacctgaaggcagaggtcacagatgaaaagctgcacgtcaccgtacgagatgcaaaaaatctaatccctatggatccaaatgggctttcggatccttacgtgaagctgaaacttattcctgaccccaagaatgagagcaaacagaaaaccaaaaccatccgatccacactgaaccctcagtggaatgagtccttcacgttcaaattaaaaccttcagacaaagaccggcgactgtccgtagaaatctgggactgggatcggacgacacggaatgacttcatgggctccctttccttcggcgtctcagagctgatgaagatgccagccagtggatggtacaagttgctcaaccaagaggagggtgaatactacaatgtgcccattccagaaggagatgaagaaggcaacgtggaactcaggcagaagttcgagaaagccaagctgggccccgctggaaacaaagtcatcagcccttcagaagacaggaagcagccatctaacaacctggacagggtgaaactcacagacttcaacttcctcatggtgctggggaaggggagttttggaaaggtgatgcttgctgacaggaagggaacagaggaactgtacgccatcaaaatcctgaagaaggacgtggtgatccaggatgacgacgtggagtgcaccatggtggagaagcgggttctggccctgctcgacaagcccccgttcctgacacagctgcactcctgcttccagacagtggaccggctgtacttcgtcatggaatacgtcaacggtggggacctcatgtaccacattcagcaagtcggaaaatttaaggagccacaagcagtattctatgcagccgagatctccatcggactgttctttcttcacaaaagaggaatcatttacagggatctgaagctggacaacgtcatgctggactcagaagggcatatcaaaatcgccgacttcgggatgtgcaaggaacacatgatggacggggtcacgaccaggaccttctgtgggactccggattacattgccccagagataatcgcttaccagccatatggaaagtctgtggactggtgggcgtacggcgtgctcctgtatgagatgctagctgggcagcttatgaccaaacaccctgccaagcggctgggctgcgggcccgagggggaaagggatgtcagagagcatgccttctttaggaggatcgactgggagaagttggagaacagggagatccaaccgccattcaagcccaaagtgtgcggcaaaggagcagaaaactttgacaagttcttcacacgagggcagcctgtcttaacaccaccagatcagctggtcatcgctaacatagaccagtctgattttgaagggttctcgtatgtcaacccccagtttgtgcacccaatcttgcaaagtgcagtatgaaactcagaaacaaaagatctaatgcctccctagcccccaatctccccagcagtgggaagtgattcttaaccataaaattttaaggctatagccttgtattttgttccacacagaggcctgaaaattctggggatattagtccataagtgatcaactttcttcccccacccaatcccaaaccaaaaaacattatcttagtggatgatgacataatatacagagtatagtttaattatgtagaagtcacatctggcttcaagttaattctttctaggaaacaaagagacttggaccctattttttggtacgatttaatatattctccatacctttcatattttggattttcactatccaaatcaaccagagataataaagtgaacccacctgaactcaagggatggaaacatttctgcccaagatatctttggaattaaagaacaggaagcccaaacagaaaacaaagagaggcagagtctcatatattcaagacctcgttgcttctattttctgcttcaatggaaacagtccctagagtctgagagggcaggatgaacctgatcactgttcccaatcatcatagcacaaccatagtgcatagtttgaaaatgaaagaaaacttcagacagatgttcgttgaatctatcatatgtactcccctgctcggttgataactatctcgataactcattctttttaagaggccaaaatcatctaaggactttgctaaacaaacatgtgaaatcatttcagatcaaggataaaccatgtgtatgttcattttaatctctgggagatgactcttcaatccagggtgccatcagtaatcatgccactgttcacgagtgttgttagccaaccccccgccacataataatattttgctacctttgtgggtacccttcctaggaagctaaaatgtatgccccatccccttttgtactatttatttaataagccgcagtgtcgtttatgaaagtacaatgtatagtaacttaatcaaagtactgactagcatcagtccctataggttgattttcctcctttctctagccccacatccacttagagatgagaaaaaaaagagtatattttgggttccaattcccagttctaattgaatggcacacatggagctgtaggatcaagacactttatgcttatatcaactcaaagatgttttcttgctagaaggattttaatatgttttgcgagtgcatcatgcaatggattttgcatgtttataataaaccttaataacaagttaatctatattattgatataattgtatcaagtataaagagtattatgataattttataagacacaattgtgctatatttgtgatgctcatgtttccagtctgctttaagattaagtgttagctttattcattgctgctggggcctctattacccgctaccccacacactggcactctatcagatatattaattttttaatgtagatgtaattttaaaatgaatggctactttacacgtttaactaggcttttactata

SEQ ID NO: 12 - DNA, 3785 bp, Rattus norvegicus
atgacgatctgtatcaagtgtgagttttgctgttttgtggttcacaagaggtgccatgagtttgttactttctcttgtccgggtgcggataagggacctgacactgatgaccccagaagcaagcacaagttcaaaatccacacctatggaagccctaccttctgtgatcactgtgggtccctgctctacggacttatccaccaagggatgaaatgcgacacctgcgacatgaatgttcacaagcagtgcgtgatcaatgtccccagcctctgcggaatggatcacacagagaagagggggcggatttacctgaaggcagaggtcacagatgaaaagctgcacgtcaccgtacgagatgcaaaaaatctaatccctatggatccaaatgggctttcggatccttacgtgaagctgaaacttattcctgaccccaagaatgagagcaaacagaaaaccaaaaccatccgatccacactgaaccctcagtggaatgagtccttcacgttcaaattaaaaccttcagacaaagaccggcgactgtccgtagaaatctgggactgggatcggacgacacggaatgacttcatgggctccctttccttcggcgtctcagagctgatgaagatgccagccagtggatggtacaagttgctcaaccaagaggagggtgaatactacaatgtgcccattccagaaggagatgaagaaggcaacgtggaactcaggcagaagttcgagaaagccaagctgggccccgctggaaacaaagtcatcagcccttcagaagacaggaagcagccatctaacaacctggacagggtgaaactcacagacttcaacttcctcatggtgctggggaaggggagttttggaaaggtgatgcttgctgacaggaagggaacagaggaactgtacgccatcaaaatcctgaagaaggacgtggtgatccaggatgacgacgtggagtgcaccatggtggagaagcgggttctggccctgctcgacaagcccccgttcctgacacagctgcactcctgcttccagacagtggaccggctgtacttcgtcatggaatacgtcaacggtggggacctcatgtaccacattcagcaagtcggaaaatttaaggagccacaagcagtattctatgcagccgagatctccatcggactgttctttcttcacaaaagaggaatcatttacagggatctgaagctggacaacgtcatgctggactcagaagggcatatcaaaatcgccgacttcgggatgtgcaaggaacacatgatggacggggtcacgaccaggaccttctgtgggactccggattacattgccccagagataatcgcttaccagccatatggaaagtctgtggactggtgggcgtacggcgtgctcctgtatgagatgctagctgggcagcctccgttcgatggcgaagacgaagatgaactgtttcagtctataatggagcacaatgtgtcctaccccaaatccttgtccaaggaagctgtctccatctgcaaagggcttatgaccaaacaccctgccaagcggctgggctgcgggcccgagggggaaagggatgtcagagagcatgccttctttaggaggatcgactgggagaagttggagaacagggagatccaaccgccattcaagcccaaagtgtgcggcaaaggagcagaaaactttgacaagttcttcacacgagggcagcctgtcttaacaccaccagatcagctggtcatcgctaacatagaccagtctgattttgaagggttctcgtatgtcaacccccagtttgtgcacccaatcttgcaaagtgcagtatgaaactcagaaacaaaagatctaatgcctccctagcccccaatctccccagcagtgggaagtgattcttaaccataaaattttaaggctatagccttgtattttgttccacacagaggcctgaaaattctggggatattagtccataagtgatcaactttcttcccccacccaatcccaaaccaaaaaacattatcttagtggatgatgacataatatacagagtatagtttaattatgtagaagtcacatctggcttcaagttaattctttctaggaaacaaagagacttggaccctattttttggtacgatttaatatattctccatacctttcatattttggattttcactatccaaatcaaccagagataataaagtgaacccacctgaactcaagggatggaaacatttctgcccaagatatctttggaattaaagaacaggaagcccaaacagaaaacaaagagaggcagagtctcatatattcaagacctcgttgcttctattttctgcttcaatggaaacagtccctagagtctgagagggcaggatgaacctgatcactgttcccaatcatcatagcacaaccatagtgcatagtttgaaaatgaaagaaaacttcagacagatgttcgttgaatctatcatatgtactcccctgctcggttgataactatctcgataactcattctttttaagaggccaaaatcatctaaggactttgctaaacaaacatgtgaaatcatttcagatcaaggataaaccatgtgtatgttcattttaatctctgggagatgactcttcaatccagggtgccatcagtaatcatgccactgttcacgagtgttgttagccaaccccccgccacataataatattttgctacctttgtgggtacccttcctaggaagctaaaatgtatgccccatccccttttgtactatttatttaataagccgcagtgtcgtttatgaaagtacaatgtatagtaacttaatcaaagtactgactagcatcagtccctataggttgattttcctcctttctctagccccacatccacttagagatgagaaaaaaaagagtatattttgggttccaattcccagttctaattgaatggcacacatggagctgtaggatcaagacactttatgcttatatcaactcaaagatgttttcttgctagaaggattttaatatgttttgcgagtgcatcatgcaatggattttgcatgtttataataaaccttaataacaagttaatctatattattgatataattgtatcaagtataaagagtattatgataattttataagacacaattgtgctatatttgtgatgctcatgtttccagtctgctttaagattaagtgttagctttattcattgctgctggggcctctattacccgctaccccacacactggcactctatcagatatattaattttttaatgtagatgtaattttaaaatgaatggctactttacacgtttaactaggcttttactatatatgtgtacatatgtatatatatatatttgattctacctgcaaaccccggttttgtttgtggggattatttttgagatgagactctgttaacttcttcatgttatcctccgttcaggttcctctaccatccctccccttggggacagctggaaatgtttaattccagtattcctgatatgttcatatatatgtggcattgttacagcacctgggctgaattccgtgtgtgtgtgtgtgtgtgtgtgtgtgtgtgtgtgtgtgtgtgtgtgtgtgtctgtgtgtctgtctgtctgtttctgtctgtttctgtctgtttctgtctgtctgtctgattcttttcagcctatgtctttggggtctctactgatgaaagggtgggaggattcctttctgagtgtgcatatggacacaacaaaaatatg

(19) United States
(12) Patent Application Publication
(10) Pub. No.: US 2023/0030314 A1
(43) Pub. Date: Feb. 2, 2023

(54) Method for Diagnosing Cancer, Cancer Diagnosis Composition, Cancer Diagnosis Kit, Method for Evaluating State of Cancer, and Method for Screening Agents for Preventing and/or Treating Cancer

(71) Applicant: Kyushu University, National University Corporation, Fukuoka-shi, Fukuoka (JP)

(72) Inventors: Masaharu Murata, Fukuoka-shi, Fukuoka (JP); Takahito Kawano, Fukuoka-shi, Fukuoka (JP); Masatoshi Eto, Fukuoka-shi, Fukuoka (JP); Junichi Inokuchi, Fukuoka-shi, Fukuoka (JP); Jeong-Hun Kang, Suita-shi, Osaka (JP)

(73) Assignee: Kyushu University, National University Corporation, Fukuoka-shi, Fukuoka (JP)

(21) Appl. No.: 17/781,796

(22) PCT Filed: Dec. 4, 2020
(86) PCT No.: PCT/JP2020/045257

§ 371 Date: Jun. 2, 2022

(30) Foreign Application Priority Data
Dec. 5, 2019 (JP) 2019-220139

(51) Int. Cl. G01N 33/574

(52) U.S. Cl. CPC G01N 33/57407; G01N 2333/9121; G01N 2440/14

(57) ABSTRACT

The present invention discloses a method for diagnosing cancer comprising a step of detecting active protein kinase Cα in urine.

### TECHNICAL FIELD

The present invention relates to a method for diagnosing cancer, a cancer diagnosis composition, a cancer diagnosis kit, a method for evaluating a state of cancer, and a method for screening agents for preventing and/or treating cancer.

### BACKGROUND ART

A living cell contains a huge number of intracellular signaling pathways for controlling or adjusting gene expression in response to extracellular signals. Phosphorylation caused by protein kinase activates a target protein in such an intracellular signaling pathway, and plays a significant role in cell growth (see Non Patent Literatures 1 to 3). Protein kinase C (PKC), that is, one of phospholipid-dependent serine/threonine kinases, is one of the most significant kinases, and is classified into three subfamilies based on the structure and features of activation. Specifically, the subfamilies are conventional or classical PKC (cPKC; α, βI, (βII, and γ), novel or non-classical PKC (nPKC; δ, ε, η, and θ), and atypical PKC (ζ, ι, and λ) (see Non Patent Literatures 4 to 6).

Among these PKC isozymes, it is known that PKCα exhibits very slight activity in a normal tissue, and is overexpressed or highly activated in most of cancer cells (for example, liver cancer, breast cancer, and melanoma) (see Non Patent Literatures 7 to 10).

### CITATION LIST

### Non Patent Literature

Non Patent Literature 1: Adjei, A. A., and Hidalgo, M. (2005) Intracellular signal transduction pathway proteins as targets for cancer therapy. J. Clin. Oncol. 23, 5386-5403.
Non Patent Literature 2: Cross, T. G., Scheel-Toellner, D., Henriquez, N. V., Deacon, E., Salmon, M., and Lord, J. M. (2000) Serine/threonine protein kinases and apoptosis. Exp. Cell Res. 256, 34-41.
Non Patent Literature 3: Bode, A. M., and Dong, Z. (2005) Signal transduction pathways in cancer development and as targets for cancer prevention. Prog. Nucleic Acid Res. Mol. Biol. 79, 237-297.
Non Patent Literature 4: Newton, A. C. (1997) Regulation of protein kinase C. Curr. Opin. Cell Biol. 9, 161-167.
Non Patent Literature 5: Blobe, G. C., Obeid, L. M., and Hannun, Y A. (1994) Regulation of protein kinase C and role in cancer biology. Cancer Metastasis Rev. 13, 411-431.
Non Patent Literature 6: Webb, B. L. J., Hirst, S. J., and Giembycz, M. A. (2000) Protein kinase C isoenzymes: a review of their structure, regulation and role in regulating airways smooth muscle tone and mitogenesis. Br. J. Pharmacol. 130, 1433-1452.
Non Patent Literature 7: Oka, M., and Kikkawa, U. (2005) Protein kinase C in melanoma. Cancer Metast. Rev. 24, 287-300.
Non Patent Literature 8: Mackay, H. J., and Twelves, C. J. (2003) Protein kinase C: a target for anticancer drugs? Endocrin. Relat. Cancer 10, 389-396.
Non Patent Literature 9: Hofmann, J (2004) Protein kinase C isozymes as potential targets for anticancer therapy. Curr. Cancer Drug Targets 4, 125-146.
Non Patent Literature 10: Goekjian, P. G., and Jirousek, M. R. (2001) Protein kinase C inhibitors as novel anticancer drugs. Expert. Opin. Investig. Drugs 10, 2117-2140.
Non Patent Literature 11: O'Brian, C. A., Chu, F., Bornmann, W. G., and Maxwell, D. S. (2006) Protein kinase Cα and ε small-molecule targeted therapeutics: a new roadmap to two holy grails in drug discovery? Expert. Rev. Anticancer Ther. 6, 175-186.

### SUMMARY OF INVENTION

### Technical Problem

An object of the present invention is to provide a novel biomarker for cancer.

### Solution to Problem

PKCα has been reported to be overexpressed or highly activated in most of cancer cells as described above, but is presumed not to be excreted in urine due to the functions of the glomerulus of the renal corpuscle. This is because PKCα is a negatively charged molecule having a molecular weight of 82 kDa, and the glomerulus of the renal corpuscle has two functions, that is, a size barrier effect of not allowing a protein molecule having a molecular weight of 60 kDa or more to pass therethrough, and a charge barrier through which a negatively charged substance cannot pass because electrically repulsed.

The present inventors found that active protein kinase Ca (active PKCα) can be detected in urine of a cancer patient contrary to the presumption, resulting in accomplishing the present invention.

Specifically, the present invention encompasses, for example, the following [1] to [9]:

[1]

A method for diagnosing cancer comprising: a step of detecting active protein kinase Ca (active PKCα) in urine.

[2]

The method according to [1], wherein the cancer includes urinary cancer.

[³]

The method according to [1] or [2], wherein the cancer includes urothelial carcinoma. [4]

The method according to any one of [1] to [3], wherein the cancer includes non-muscle-invasive cancer.

[5]

A cancer diagnosis composition comprising: an antibody or a substrate peptide for detecting active PKCα, wherein diagnosis is made using urine of an examinee.

[6]

A cancer diagnosis kit comprising: an antibody or a substrate peptide for detecting active PKCα, wherein diagnosis is made using urine of an examinee.

[7]

A method for evaluating a state of cancer, wherein active PKCα is detected by reacting an antibody to the active PKCα with urine of an examinee, and a state of the cancer is evaluated with an obtained detection result used as an index.

[8]

A method for evaluating a state of cancer by using, as an index, a detection result obtained by reacting a substrate peptide of active PKCα with urine of an examinee, and detecting the peptide having been phosphorylated.

[9]

A method for screening agents for preventing and/or treating cancer comprising: a step of administering a candidate substance to a urinary cancer model non-human mammal; a step of detecting active PKCα in urine of the non-human mammal; and a step of selecting an intended agent for preventing and/or treating cancer with an obtained result used as an index.

### Advantageous Effects of Invention

According to the present invention, a method for diagnosing cancer, a cancer diagnosis composition, a cancer diagnosis kit, a method for evaluating a state of cancer, and a method for screening agents for preventing and/or treating cancer, in which active PKCα in urine is used as a biomarker, can be provided. The present invention uses a biomarker present in urine, and hence is simple and non-invasive. As described below, specificity against and sensitivity to early stage cancer are also high.

### BRIEF DESCRIPTION OF DRAWINGS

FIG. 1 is a diagram illustrating MALDI-TOF MS spectra obtained as a result of analysis by MALDI-TOF MS of a phosphorylation reaction of a substrate peptide in bladder cancer-derived cultured cells (KU-1, KU-7, T24, TCCSUP, and UMUC-3).

FIG. 2 is a graph illustrating a phosphorylation percentage (%) of the substrate peptide in the bladder cancer-derived cultured cells (KU-1, KU-7, T24, TCCSUP, and UMUC-3).

FIG. 3 illustrates case information on urothelial carcinoma patients. Each gray cell indicates that a corresponding result is positive or a corresponding numerical value is equal to or larger than a threshold. “Histological type (primary)” indicates a histological type (primary) of cancer, “urinalysis (RBC)” indicates urinalysis (erythrocyte), “urinalysis (WBC)” indicates urinalysis (leukocyte), and “urine cytology” indicates urine cytology.

FIG. 4 illustrates case information on non-urothelial carcinoma patients. Each gray cell indicates that a corresponding result is positive or a corresponding numerical value is equal to or larger than a threshold. “Urinalysis (RBC)” indicates urinalysis (erythrocyte), and “urinalysis (WBC)” indicates urinalysis (leukocyte).

FIG. 5 is a histogram illustrating phosphorylation percentages of a substrate peptide in urine samples obtained from urothelial carcinoma patients and non-urothelial carcinoma patients. A line with white circles indicate data of the urothelial carcinoma patients, and a line with black circles indicate data of the non-urothelial carcinoma patients.

FIG. 6 is a graph illustrating, in the form of a probability density function, the relationship between the phosphorylation percentages and frequencies of a substrate peptide in urine samples obtained from urothelial carcinoma patients and non-urothelial carcinoma patients. A line with white circles indicates data of the urothelial carcinoma patients, and a line with black circles indicates data of the non-urothelial carcinoma patients.

FIG. 7 is a graph illustrating, in the form of a cumulative distribution function, the relationship between the phosphorylation percentages and frequencies of a substrate peptide in urine samples obtained from urothelial carcinoma patients and non-urothelial carcinoma patients. A line with white circles indicates data of the urothelial carcinoma patients, and a line with black circles indicates data of the non-urothelial carcinoma patients.

FIG. 8 is a graph illustrating the phosphorylation percentages of a substrate peptide in urine samples obtained from low grade urothelial carcinoma patients and high grade urothelial carcinoma patients (*: P<0.05). A dotted line indicates a cutoff value (phosphorylation percentage of 2%).

FIG. 9 illustrates receiver operating characteristic (ROC) curves of diagnosis based on the phosphorylation percentages of a substrate peptide using urine samples. A dashed line indicates an ROC curve of low grade urothelial carcinoma patients, and a solid line indicates an ROC curve of high grade urothelial carcinoma patients.

### DESCRIPTION OF EMBODIMENTS

In the present invention, based on finding that active PKCα is present in urine of a cancer patient, the active PKCα present in urine is used as a cancer marker.

[Active PKCα]

Active PKCα means PKCα activated by phosphorylating the 497th threonine residue, the 638th threonine residue, and the 657th serine residue in human PKCα, or corresponding threonine residues and serine residue in PKCα of another animal

[Target Cancer]

Cancer (type of cancer) to be diagnosed or evaluated is not especially limited, and two or more types of cancer can be targets of diagnosis and evaluation. Target cancer preferably includes urinary cancer. Examples of urinary cancer include tumors in the renal parenchyma, the urinary tract including the renal pelvis, the ureter, the bladder, and the urethra, and the genitals such as the prostate, the seminal vesicle, the penis, and the testicular, and representative examples include urothelial carcinoma, prostate cancer, kidney cancer, and testicular cancer.

The target cancer may include urothelial carcinoma. Examples of urothelial carcinoma include bladder cancer, ureteral cancer, urethral cancer, and renal pelvis cancer. The target cancer may be selected from the group consisting of bladder cancer, ureteral cancer, urethral cancer, and renal pelvis cancer.

Urothelial carcinoma refers to cancer occurring in a cell of the mucosal urinary tract designated as urothelium (transitional epithelium) present inside the urinary tract extending from the renal pelvis to the ureter, the bladder, and a part of the urethra. Principal one of urothelial carcinomas is bladder cancer, and the tumor grade of bladder cancer is evaluated in two stages of low grade and high grade, or in three stages of G1 to G3, and the tumor grade is higher and malignancy is higher in high grade and G3. Tumor depth (T stage) is classified as described below, and Ta to T1 are designated as non-muscle-invasive cancer, among which Tis is designated as carcinoma in situ, and T2 and higher stages corresponding to muscle-invasive cancer (“General Rule for Clinical and Pathological Studies on Renal Pelvis, Ureteral and Bladder Cancer), April 2011 (first edition)” edited by The Japanese Urological Association, The Japanese Society of Pathology, and Japan Radiological Society (Kanehara & Co., Ltd.)).

TX: Primary tumor cannot be assessed

T0: No evidence of primary tumor

Ta: Non-invasive papillary cancer

Tis: Carcinoma in situ (CIS)

T1: Tumor invading subepithelial connective tissue

T2a: Invasion into central portion of muscle

T2b: Invasion beyond central portion of muscle

T3a: Microscopic invasion into adipose tissue around bladder presumed

T3b: Macroscopically definite extramural invasion into adipose tissue around bladder presumed

T4a: Invasion into prostate, seminal vesicle, uterine, or vagina

T4b: Invasion into pelvic wall or abdominal wall

Currently, Nuclear matrix protein 22 (NMP22), Bladder tumor antigen test (BTA test) and the like are used as cancer biomarkers, but there is a problem that all of these markers have low sensitivity as a bladder cancer marker (particularly sensitivity to low grade cancer). On the contrary, a marker utilizing active PKCα in urine of the present invention exhibits a high specificity against and a high sensitivity to also non-muscle-invasive cancer, particularly low grade cancer.

Accordingly, the target cancer may be, for example, non-muscle-invasive cancer, may be low grade cancer, and may be carcinoma in situ.

[Method for Diagnosing Cancer]

A method of the present embodiment provides an index for diagnosing that a subject (examinee) has cancer based on that active PKCα is present in urine of a cancer patient. The method for diagnosing cancer of the present embodiment includes a step of detecting active PKCα in urine.

The method of the present embodiment may further include a step of collecting urine (urine specimen) corresponding to a test sample. Urine can be collected, for example, with a urine collecting container. Urine collected from a subject can be used. The subject may be a human, or may be a non-human mammal. The subject may be an animal suspected to have cancer.

The method of the present embodiment may include a step of appropriately pretreating the urine specimen in accordance with a method for detecting active PKCα. Examples of such a pretreatment include centrifugation, heating, addition of a surfactant, cell disruption with a homogenizer or an ultrasonic cell disruptor, dilution with a sample buffer or the like, and a treatment with a protease inhibitor.

The method of the present embodiment can provide an index for diagnosing cancer, for example, based on an amount of active PKCα present in urine. Examples of the index for diagnosing cancer include an index of having onset of cancer or a high possibility of future onset, and on the contrary, an index of having a low possibility of onset of cancer, or a low possibility of future onset. For example, that an amount of active PKCα in urine of a subject is larger than a reference value may indicate that the subject has cancer or has a high possibility of future onset. The reference value may be a value set based on the amount of active PKCα in urine of a healthy person, may be a value set based on the amount of active PKCα in urine of a cancer patient, or may be a value set by comparison between the amount of active PKCα in urine of a healthy person and the amount of active PKCα in urine of a cancer patient. The reference value may be, for example, a value for directly comparing the amount of active PKCα of the subject, or may be a value set as a factor (for example, 10-fold) in comparison with the amount of active PKCα in urine of a healthy person. The reference value may be a value set based on a plurality of samples, such as an average. Here, the term “healthy person” used in the above description may be replaced with a person not having cancer (non-cancer patient), and a non-cancer patient may be a person having been diagnosed not to have specific cancer (such as urinary cancer).

In addition, the method of the present embodiment can be used for determining a state (disease condition) of cancer by periodically monitoring the amount of active PKCα of one subject. For example, an increase trend of the amount of active PKCα in urine of a subject may indicate onset of cancer or a high possibility of future onset.

A method for detecting active PKCα in urine of a subject is not especially limited, and can be performed by a known method. For example, since active PKCα has a phosphorylation function, urine of a subject can be reacted with a substrate peptide of active PKCα to detect the peptide having been phosphorylated. Alternatively, for example, an antibody to active PKCα is reacted with urine to directly detect active PKCα. Accordingly, the step of detecting active PKCα in urine may include, for example, reacting a substrate peptide of active PKCα with the urine to detect the peptide having been phosphorylated, or may include reacting an antibody to active PKCα with the urine to detect PKCα.

[Detection with Substrate Peptide]

When the step of detecting active PKCα in urine includes reacting a substrate peptide of active PKCα with the urine to detect the peptide having been phosphorylated, that the detected amount of the phosphorylated PKCα substrate peptide is larger than a reference value may indicate that the subject has cancer. The reference value may be, for example, set based on an amount of phosphorylated PKCα substrate peptide detected in urine of a healthy person, may be a value set based on an amount of phosphorylated PKCα substrate peptide detected in urine of a cancer patient, or may be a value set by comparison between the amount of phosphorylated PKCα substrate peptide detected in urine of a healthy person and the amount of phosphorylated PKCα substrate peptide detected in urine of a cancer patient. The reference value may be, for example, a value for directly comparing the amount of phosphorylated PKCα substrate peptide detected in a subject, or may be a value set as a factor (for example, 10-fold) in comparison with the amount of phosphorylated PKCα substrate peptide detected in urine of a healthy person. The reference value may be a value set based on a plurality of samples, such as an average. When the amount of phosphorylated PKCα substrate peptide is quantitatively determined by obtaining a phosphorylation percentage to the amount of PKCα substrate peptide used in the reaction as described below, the phosphorylation percentage may be used as the reference value. The phosphorylation percentage used as the reference value may be set, for example, in a range of 1 to 7%, 1.5% to 5%, 1.5 to 4%, or 2% to 3%.

The substrate peptide of active PKCα is not limited but may be any peptide specifically phosphorylated by PKCα, and an example includes a substrate peptide consisting of 10 to 30 amino acid residues (preferably 10 to 20 amino acid residues, and more preferably 10 to 15 amino acid residues). Specific examples of the peptide preferably include a peptide consisting of an amino acid sequence (FKKQGSFAKKK) of SEQ ID NO: 1, and a peptide consisting of an amino acid sequence (FKKQGTFAKKK) of SEQ ID NO: 2. It is noted that the peptide consisting of the amino acid sequence of SEQ ID NO: 1 is herein designated also as “Alphatomega”.

The PKCα substrate peptide can be synthesized, for example, with an automated peptide synthesizer in accordance with standard Fmoc chemistry procedures. After the synthesis, the resultant can be purified by a known purification method using various chromatography or the like.

In the method of the present embodiment, the amount of the PKCα substrate peptide to be used can be appropriately set by those skilled in the art, and may be, for example, 1 to 1000 μM (preferably 1 to 500 μM, and more preferably 1 to 50 μM).

(i) Detection of Phosphorylated PKCα Substrate Peptide

Urine collected as a test sample may be subjected to an appropriate pretreatment such as centrifugation, heating, addition of a surfactant, cell disruption with a homogenizer or an ultrasonic cell disruptor, dilution with a sample buffer or the like, or a treatment with a protease inhibitor. When the dilution is performed, as the sample buffer, for example, HEPES (4-(2-hydroxyethyl)-1-piperazineethanesulfonic acid) buffer, phosphate buffered saline, or the like can be used.

After performing the pretreatment if necessary, the test sample is contacted with the PKCα substrate peptide, and when active PKCα is present in the test sample, the PKCα substrate peptide is phosphorylated by its phosphorylation function. In the phosphorylation reaction, a reaction medium is not especially limited, and for example, a buffer obtained by mixing Tris-HCl and magnesium chloride or the like can be used, and as a phosphate donating substrate, for example, a mixture of adenosine triphosphate (ATP) or the like with the buffer can be used.

The reaction between the test sample and the PKCα substrate peptide is performed at 30 to 40° C. (preferably 37° C.) for 10 to 60 minutes (preferably 30 to 60 minutes).

The detection of the phosphorylated PKCα substrate peptide can be performed, although not limited thereto, by employing known immunoassay in accordance with the ordinary method. Examples of the immunoassay include labeled immunoassay and turbidimetric immunoassay (TIA), and examples include enzyme immunoassay (EIA) such as ELISA, radioimmunoassay (RIA) and fluoroimmunoassay (FIA). Examples of the labeled immunoassay combined with another separation method include Western blotting (combination with electrophoresis). Among these methods, ELISA and/or Western blotting is preferred. Alternatively, one or two or more monoclonal antibodies to the phosphorylated PKCα substrate peptide may be used to perform the detection with a combination of two or more immunoassays. Thus, excellent detection with further higher sensitivity and reliability can be performed.

As the method for detecting phosphorylated PKCα substrate peptide, a method using a metal colloid such as a metal nanoparticle can be also employed. Specifically, this method is a method based on principle that when a substrate peptide and protein kinase are reacted with each other in the presence of a metal colloid, or when a metal colloid is added after a reaction between a substrate peptide and protein kinase, the level of phosphorylation of the substrate peptide by protein kinase can be evaluated based on color change of the metal colloid.

Regarding this color change, when a metal colloid is caused to present in the phosphorylation reaction of the substrate peptide by protein kinase, the phosphorylated substrate peptide is agglomerated due to the presence of the metal colloid particle, and the color of the metal colloid is changed based on the degree of agglomeration. The color of the metal colloid changes in accordance with the degree of agglomeration substantially in proportion to phosphorylation of the substrate peptide.

Accordingly, a reaction between the test sample and the PKCα substrate peptide is performed in the presence of a metal colloid, or the metal colloid is added after the reaction, and color change of the sample is compared with that of the sample before the reaction, or compared with color change of a control (reaction with a sample collected from a healthy person), and thus, the presence of phosphorylation of the PKCα substrate peptide can be detected.

In the method using the metal colloid, as a reaction medium, for example, a buffer obtained by mixing HEPES (4-(2-hydroxyethyl)-1-piperazineethansulfonic acid) and magnesium chloride, or the like is used, and as a phosphate donating substrate, ATP or the like is used in the same manner as described above.

As the metal colloid of metal nanoparticles or the like, for example, a gold colloid, a silver colloid, a platinum colloid, an iron colloid, an iron hydroxide colloid, an aluminum hydroxide colloid, a palladium colloid, a rhodium colloid or the like is used. Among these metal colloids, a gold colloid and a silver colloid are preferred, and a gold colloid is more preferred. These metal colloids can be produced by methods described in known literatures, or a commercially available product can be used. For example, a gold nanoparticle can be generally produced by a liquid phase reduction method in which a reducing solution is added to a solution containing a gold ion to reduce the gold ion (see, for example, Japanese Unexamined Patent Publication No. 2003-253310, and Japanese Unexamined Patent Publication No. 2006-152438).

A particle size of the metal colloid is not especially limited, and for example, is preferably about 1 nm to 500 nm, more preferably 3 nm to 200 nm, and further preferably 5 nm to 100 nm.

Although the color of a metal colloid depends on the particle size, a gold colloid is usually red, and a silver colloid is yellow. On the contrary, for example, when a gold colloid is present, phosphorylation and agglomeration of the PKCα substrate peptide results in changing the color of the gold colloid from red to blue. Based on such color change, the phosphorylated PKCα substrate peptide can be detected. It is noted that the color change of a metal colloid is checked by measuring an absorbance preferably in a range of about 600 nm to 800 nm, more preferably in a range of about 600 nm to 750 nm, and further preferably at about 700 nm.

(ii) Quantitative Determination of Phosphorylated PKCα Substrate Peptide

Although not limited thereto, quantitative determination can be performed by obtaining a phosphorylation percentage to PKCα substrate peptide used in the reaction (ionic strength ratio between a phosphorylated substrate and a non-phosphorylated substrate) by employing matrix-assisted laser desorption/ionization time-of-flight mass spectrometry (MALDI-TOF MS).

In addition, when the phosphorylated PKCα substrate peptide is detected by the method using the metal colloid, the phosphorylated substrate peptide can be substantially quantitatively determined based on the degree of color change of the metal colloid (specifically, by comparing the degree of color change obtained with an appropriate control, or with a precedently created calibration curve).

The quantitative determination or detection of the phosphorylated PKCα substrate peptide can be performed with a commercially available kit.

[Detection with Antibody]

When the step of detecting active PKCα in urine includes reacting an antibody to active PKCα with the urine to detect the PKCα, that a detected amount of active PKCα is larger than a reference value may indicate that the subject has cancer. The reference value may be a value set based on an amount of active PKCα detected in urine of a healthy person, may be a value set based on an amount of active PKCα detected in urine of a cancer patient, or may be a value set by comparison between an amount of active PKCα detected in urine of a healthy person and an amount of active PKCα detected in urine of a cancer patient. The reference value may be, for example, a value for directly comparing the detected amount of active PKCα of the subject, or may be a value set as a factor (for example, 10-fold) in comparison with the amount of active PKCα detected in urine of a healthy person. The reference value may be a value set based on a plurality of samples, such as an average.

An antibody to active PKCα (anti-PKCα antibody) can be produced as follows.

(A) Production of Polyclonal Antibody

(i) Preparation of Antigen and Solution Thereof

For producing an anti-PKCα antibody, it is first necessary to prepare or obtain a protein to be used as an immunogen (antigen).

An antigen protein can be, but is not limited to, purified PKCα, and for example, a protein having an amino acid sequence obtained by deleting, substituting or adding one or several amino acids in the amino acid sequence of PKCα, and having PKCα activity can be used. It is noted that, for example, PKCα derived from a mammal, specifically, human-derived PKCα, mouse-derived PKCα, or rat-derived PKCα, can be used as the PKCα. Here, nucleotide sequence and amino acid sequence information of PKCα derived from each animal is available as the following Accession Nos.

<Human-Derived PKCα>
GenBank Accession No: NM 002737 (nucleotide sequence: SEQ ID NO: 3), NP 002728 (amino acid sequence: SEQ ID NO: 4)
GenBank Accession No: X52479 (nucleotide sequence: SEQ ID NO: 5), CAA36718 (amino acid sequence: SEQ ID NO: 6)

<Mouse-Derived PKCα>
GenBank Accession No: NM 011101 (nucleotide sequence: SEQ ID NO: 7), NP 035231 (amino acid sequence: SEQ ID NO: 8)
GenBank Accession No: M25811 (nucleotide sequence: SEQ ID NO: 9), AAA39934 (amino acid sequence: SEQ ID NO: 10)

<Rat-Derived PKCα>
GenBank Accession No: NM 001105713.1 (nucleotide sequence: SEQ ID NO: 11), XM 343975 (nucleotide sequence: SEQ ID NO: 12)

A method for preparing purified PKCα to be used as an antigen is not limited, and for example, a method in which PKCα is produced using a transformant of an appropriate host cell (such as a human-derived cultured fibroblast, a Chinese hamster ovary cell, or yeast) for preparation by a general biochemical method employed for isolation purification of a protein, for example, one of or a combination of ammonium sulfate precipitation, gel chromatography, ion exchange chromatography, and affinity chromatography can be employed.

Next, the purified PKCα thus obtained is dissolved in a buffer to prepare an antigen solution. At this point, an adjuvant for effective immunization may be added if necessary. As the adjuvant, for example, commercially available Freund's complete adjuvants or Freund's incomplete adjuvants can be used. One of such adjuvants may be used, or two or more of these may be used together, which is not restrictive.

(ii) Immunization, and Collection of Anti-serum

Immunization is performed by administering a solution containing the purified PKCα to a mammal (such as a mouse, a rat, and a rabbit). The administration is performed mainly by intravenous, subcutaneous, or intraperitoneal injection.

A single dose of the antigen solution is not limited, and for example, preferably contains the purified PKCα of 2 to 500 μg, and more preferably 10 to 100 μg per animal

A dosage interval is not limited, and for example, is preferably an interval of several days to several weeks, and more preferably an interval of 2 to 3 weeks. The number of doses is, for example, 2 to 10.

Although not limited thereto, a serum obtained by the immunization (anti-serum) is collected, for example, preferably 1 to 28 days, and more preferably 2 to 14 days after a day of the final administration. As a method for collecting an anti-serum, the anti-serum can be collected from blood of the immunized animal by an ordinary method.

(iii) Sorting of Intended Anti-serum

From the anti-serums collected from respective immunized animals, an intended anti-serum, namely, an anti-serum containing an anti-PKCα antibody, is screened.

A screening method is not limited, and screening can be performed, for example, by employing any of known immunoassays in accordance with an ordinary method using the collected anti-serum, and the purified PKCα used as the antigen or recombinant PKCα (produced in a human-derived cultured fibroblast, a Chinese hamster ovary cell, or yeast), or a mutant enzyme thereof. Examples of the immunoassays include labeled immunoassay and turbidimetric immunoassay (TIA), and the former is preferred, and preferable examples include enzyme immunoassay (EIA) such as ELISA, radioimmunoassay (RIA) and fluoroimmunoassay (FIA). As the labeled immunoassay, preferable examples of its combination with another separation method include Western blotting (combination with electrophoresis). It is noted that since a protein sample to be detected is denatured with heat and a surfactant in Western blotting, an antigen to be used in the screening method is a mutant enzyme.

The anti-PKCα antibody contained in the intended anti-serum obtained by the screening is usually a polyclonal antibody. When the antibody needs to be purified, purification can be performed by appropriately selecting, for example, only one of, or a combination of two or more of known purification methods, such as ammonium sulfate salting-out method, ion exchange chromatography, affinity chromatography, and gel chromatography.

A reactive site in PKCα to which the anti-PKCα antibody can specifically bind (specifically recognize) is not especially limited.

(B) Production of Monoclonal Antibody

(i) Preparation of Antigen and Solution Thereof

Preparation of an antigen and a solution thereof can be performed in the same manner as in the production of the polyclonal antibody described above.

(ii) Immunization, and Collection of Antibody-Producing Cell

An immunization method, as well as a dose, a dosage interval, and the number of doses using the antigen solution can adopt the same method and conditions employed in the production of the polyclonal antibody described above.

Although not limited thereto, a cell producing the anti-PKCα antibody (antibody-producing cell) obtained by the immunization is collected, for example, preferably 1 to 14 days, and more preferably 2 to 4 days after a day of the final administration.

Preferable examples of the antibody-producing cell include a spleen cell, a lymph node cell (particularly, a regional lymph node cell), and a peripheral blood cell, and in particular, a spleen cell and a regional lymph node cell (such as a popliteal lymph node cell) are more preferred.

(iii) Cell Fusion

Cell fusion of the thus collected antibody-producing cell and a myeloma cell is performed, and thus, a fused cell (hybridoma) can be obtained.

As the myeloma cell, an established cell generally available in a mammal such as a mouse can be used. In more detail, an established cell that has drug selectivity, and has a property that it cannot grow in a HAT selective medium (hypoxanthine-, aminopterin- and thymine-containing medium) in an unfused state but can grow in a state fused with the antibody-producing cell is preferred. Specifically, as a mouse myeloma cell, for example, PAI, P3X63-Ag.8.U1 (P3U1), NS—I and the like can be used.

The cell fusion is performed, for example, by mixing the antibody-producing cell and the myeloma cell and causing a fusion reaction therebetween in an animal cell culture medium such as serum-free RPMI-1640 medium. A mixing ratio between the antibody-producing cell and the myeloma cell is, for example, 5:1.

The fusion reaction is performed preferably in the presence of a cell fusion promoter in general, and as the promoter, polyethylene glycol having an average molecular weight of 1000 to 6000 Dalton, or the like can be used. Alternatively, a commercially available cell fusion device utilizing electrical stimulation (such as electroporation) can be used for fusing the antibody-producing cell and the myeloma cell.

The cell resulting from the fusion reaction is cultured in, for example, HAT selective medium. After the culture, a cell found to grow in the HAT selective medium corresponds to the fused cell (hybridoma).

(iv) Sorting of Intended Hybridoma, and Cloning thereof

From hybridomas obtained through the above-described culture, an intended hybridoma, namely, a hybridoma producing the anti-PKCα antibody is screened. Specifically, a culture supernatant containing the intended anti-PKCα antibody is screened.

A screening method is not limited, and for example, the screening can be performed by employing any of known immunoassays in accordance with an ordinary method using a part of the culture supernatant, and the purified PKCα used as the antigen or recombinant PKCα (produced in a human-derived cultured fibroblast, a Chinese hamster ovary cell, or yeast), or a mutant enzyme thereof. Examples of the immunoassays include enzyme immunoassay (EIA) such as ELISA, radioimmunoassay (RIA) and fluoroimmunoassay (FIA). Western blotting (combination with electrophoresis) or the like is also preferably employed. It is noted that since a protein sample to be detected is denatured with heat and a surfactant in Western blotting, an antigen to be used in the screening method is a mutant enzyme.

The anti-PKCα antibody contained in a culture supernatant of the intended hybridoma resulting from the screening is the antibody obtained before cloning the hybridoma, and may be an antibody containing a single molecule (monoclonal antibody), which is not restrictive.

A reactive site in PKCα to which the anti-PKCα antibody can specifically bind (specifically recognize) is not especially limited, and for example, it may be an antibody specifically recognizing a site where PKCα is phosphorylated (such as sites of Thr497, Thr638, and Ser657). When an antibody specifically recognizing a site where PKCaa is phosphorylated is used, detection accuracy of active PKCα can be expected to be improved.

Cloning of the intended hybridoma by the screening, namely, establishment of a monoclonal antibody-producing cell line, can be performed generally by selecting a colony derived from a single cell through culture by a limiting dilution method or the like.

(v) Collection of Monoclonal Antibody

A method for collecting the monoclonal antibody from the hybridoma resulting from the cloning is not limited, and a cell culture method, or an ascites formation method or the like can be generally employed.

In the cell culture method, the hybridoma resulting from the cloning is cultured, for example, in an animal cell culture medium under conditions of 37° C. and 5% CO₂ for 7 to 14 days, and the intended monoclonal antibody can be collected from a culture supernatant thus obtained.

In the ascites formation method, for example, the hybridoma resulting from the cloning is administered, in an amount of 10⁶ cells per animal, intraperitoneally to an animal of the same species as the mammal from which the myeloma cell used in the cell fusion is derived, and thus, the hybridoma is grown in a large amount, and the intended monoclonal antibody can be collected from ascites fluid or serum collected 7 to 14 days after the administration.

In employing either of the cell culture method and the ascites formation method, when the monoclonal antibody needs to be purified in or after collecting the antibody, only one of, or a combination of two or more of known purification methods, such as ammonium sulfate salting-out method, ion exchange chromatography, affinity chromatography, and gel chromatography can be appropriately selected and performed.

In the method of the present embodiment, an amount of the anti-PKCα antibody to be used can be appropriately set by those skilled in the art, and may be, for example, with 1 μg assumed as a standard, 1 μg to 10 μg, 1 μg to 100 μg or 1 μg to 1000 μg.

(C) Detection and Quantitative Determination of Active PKCα

(i) Detection of active PKCα

Urine collected as a test sample may be subjected to an appropriate pretreatment such as centrifugation, heating, addition of a surfactant, cell disruption with a homogenizer or an ultrasonic cell disruptor, dilution with a sample buffer or the like, or a treatment with a protease inhibitor. When the dilution is performed, as the sample buffer, for example, HEPES (4-(2-hydroxyethyl)-1-piperazineethanesulfonic acid) buffer, phosphate buffered saline, or the like can be used.

After performing the pretreatment if necessary, the test sample is reacted with the anti-PKCα antibody (preferably a monoclonal antibody in general), and when the test sample contains active PKCα, an antigen-antibody reaction occurs.

The reaction between the test sample and the anti-PKCα antibody is performed at 20 to 40° C. (preferably 30 to 37°) for 30 to 720 minutes (preferably 30 to 90 minutes).

The detection of active PKCα can be performed by employing any of known immunoassays in accordance with an ordinary method thereof. Examples of the immunoassays include labeled immunoassay and turbidimetric immunoassay (TIA), and the former is preferred, and preferable examples include enzyme immunoassay (EIA) such as ELISA, radioimmunoassay (RIA) and fluoroimmunoassay (FIA). As the labeled immunoassay, preferable examples of its combination with another separation method include Western blotting (combination with electrophoresis). Among these methods, ELISA and/or Western blotting is preferred. In addition, one or two or more anti-PKCα monoclonal antibodies may be used, and the detection may be performed by a combination of two or more immunoassays. Thus, excellent detection with still higher sensitivity and reliability can be performed.

(ii) Quantitative Determination of Active PKCα

Although quantitative determination is not limited, active PKCα contained in a test sample is quantitatively determined preferably from a calibration curve based on the relationship between an antigen level (antigen concentration: PKCα concentration) and a detected value thereof. The calibration curve is precedently created based on detected values (detection data) obtained by using purified PKCα as the antigen and using the anti-PKCα antibody. In more detail, information of detected values against the antigen concentration is obtained by a method similar to the immunoassay (such as ELISA or Western blotting) described above as the detection method, and the calibration curve is created based on the information. When the calibration curve is compared with an actually detected value (measured value), an amount of active PKCα in the test sample can be obtained. The quantitative determination or detection of PKCα can be performed with a commercially available kit.

[Cancer Diagnosis Composition]

A cancer diagnosis composition of the present embodiment contains an antibody or a substrate peptide for detecting active PKCα, and is characterized by that diagnosis is made using urine of an examinee.

As the antibody or the substrate peptide for detecting active

PKCα, those described above can be used. The cancer diagnosis composition of the present embodiment is used for detecting and quantitatively determining active PKCα in urine of an examinee, and can be used for the diagnosis described above. A method for detecting active PKCα and the like are the same as those described above.

A compounding ratio of the antibody in the cancer diagnosis composition is not especially limited, and for example, may be 1 to 100% by weight, 5 to 100% by weight, 10 to 100% by weight, 3 to 99% by weight, 5 to 99% by weight, 10 to 99% by weight, 10 to 95% by weight, or 20 to 95% by weight based on the total weight of the composition. Although not limited thereto, the cancer diagnosis composition of the present embodiment may contain, in addition to the anti-PKCα antibody, another constituent component as long as the effects of the present invention are largely impaired. For example, a primary antibody detection reagent, a chromogenic substrate and the like can be contained.

A compounding ratio of the PKCα substrate peptide in the cancer diagnosis composition is not especially limited, and for example, may be 1 to 100% by weight, 5 to 100% by weight, 10 to 100% by weight, 3 to 99% by weight, 5 to 99% by weight, 10 to 99% by weight, 10 to 95% by weight, or 20 to 95% by weight based on the total weight of the composition. Although not limited thereto, the cancer diagnosis composition of the present embodiment may contain, in addition to the PKCα substrate peptide, another constituent component as long as the effects of the present invention are largely impaired. For example, an antibody to the phosphorylated PKCα substrate peptide or the like can be contained.

[Cancer Diagnosis Kit]

A cancer diagnosis kit of the present embodiment includes an antibody or a substrate peptide for detecting active PKCα, and is characterized by that diagnosis is made using urine of an examinee.

As the antibody or the substrate peptide for detecting active PKCα, those described above can be used. The cancer diagnosis kit of the present embodiment is used for detecting and quantitatively determining active PKCα in urine of an examinee, and can be used for the diagnosis described above. A method for detecting active PKCα and the like are the same as those described above.

The cancer diagnosis kit of the present embodiment may include the cancer diagnosis composition described above.

When the cancer diagnosis kit includes the anti-PKCα antibody, the antibody is preferably provided in a dissolved state in consideration of stability (preservability), ease of use, and the like.

When the cancer diagnosis kit includes the PKCα substrate peptide, the substrate peptide is preferably provided in a state of a power or in a state of a solution (state dissolved in pure water, a buffer or a saline) and stored under conditions of cold storage or frozen storage (−30° C. to −80° C.) in consideration of stability (preservability), ease of use, and the like.

The kit can include, in addition to the PKCα substrate peptide or the anti-PKCα antibody, another constituent element. Examples of another constituent element include a primary antibody detection reagent, a chromogenic substrate or the like that can be used in ELISA or Western blotting.

The kit can be any kit including, as a constituent element, at least the PKCα substrate peptide or the anti-PKCα antibody. Accordingly, all the constituent elements essential for cancer diagnosis may be, or may not be included in addition to the PKCα substrate peptide or the anti-PKCα antibody, which is not restrictive.

The kit may include means for collecting urine, such as a urine collecting container.

The kit may be provided with instructions for use of the kit to detect active PKCα in urine of an examinee for cancer diagnosis.

[Method for Evaluating State of Cancer]

In a method for evaluating a state of cancer of the present embodiment, active PKCα is detected by reacting an antibody to active PKCα with urine of an examinee, and a state of cancer is evaluated by using, as an index, a detection result thus obtained. Further, in a method for evaluating a state of cancer of another embodiment, a substrate peptide of active PKCα is reacted with urine of an examinee to detect the peptide having been phosphorylated, and a state of cancer is evaluated by using, as an index, a detection result thus obtained.

As the antibody or the substrate peptide for detecting active PKCα, those described above can be used. A detection method by reaction with urine of an examinee is also the same as that described above.

By the method for evaluating a state of cancer of the present embodiment, a state of cancer can be evaluated by using, as an index, a detection result (detected amount) of active PKCα or the phosphorylated substrate peptide detected or quantitatively determined. In other words, by detecting active PKCα by reacting an antibody to active PKCα with urine of an examinee, or by detecting a phosphorylated substrate peptide by reacting the PKCα substrate peptide with urine, a state of cancer is evaluated by using, as an index, a detection result thus obtained.

Here, to “evaluate a state of cancer” means that onset, progression, severity, response to treatment, prognosis or the like of cancer is determined. The evaluation may be performed based on a detected amount of active PKCα or a detected amount of phosphorylated PKCα substrate peptide, and in addition, subjective symptom and the like may be used in combination. For example, the evaluation can be performed by measuring active PKCα or phosphorylated PKCα substrate peptide at a frequency of once or twice a year as in routine medical examination, but it is preferable to comprehensively evaluate a state of cancer (including monitoring of recurrence of cancer) by periodically monitoring transition of the detected amount of active PKCα or the phosphorylated substrate peptide. As such an evaluation method, it is possible, for example, to diagnose that there is a high possibility of onset of any cancer when the detected amount of active PKCα or the phosphorylated PKCα substrate peptide is over a prescribed value, to diagnose that treatment is effective when the detected amount of active PKCα or the phosphorylated substrate peptide is reduced in a cancer case under treatment, or to diagnose that treatment and recovery are impossible in a case where the detected amount of active PKCα or the phosphorylated substrate peptide is over a prescribed value and averaged at a high value.

[Method for Screening Agents for Preventing and/or Treating Cancer]

A method for screening agents for preventing and/or treating cancer of the present embodiment includes: (a) a step of administering a candidate substance to a urinary cancer model non-human mammal (administration step); (b) a step of detecting active PKCα in urine of the non-human mammal (detection step); and (c) a step of selecting an intended agent for preventing and/or treating cancer with a result thus obtained used as an index (evaluation step). It is noted that the screening method of the present embodiment may include another step if necessary.

These steps will now be described.

(a) Administration Step

A urinary cancer model non-human mammal used as a subject animal, and a control animal are not especially limited, and from the viewpoint that an accurate comparative experiment can be performed, it is usually preferable to use non-human mammals of the same species, more preferable to use a litter of non-human mammals, and further preferable to use non-human mammals of the same sex and the same age. The subject animal and the control animal are preferably raised under the same conditions except for feed intake.

As the subject animal, a urinary cancer model non-human mammal obtained by causing cancer in a normal non-human mammal by transplantation of a tumor tissue or tumor cell by a known method can be used. Furthermore, a non-human mammal obtained by surgically resecting the tumor tissue or tumor cell from the urinary cancer model non-human mammal can be used. When the non-human mammal obtained after resection is used as the subject animal, for example, a method for screening agents for preventing recurrent cancer can be performed.

The control animal is not limited as long as it is suitable to be used as a comparative control of the subject animal, and for example, a normal non-human mammal in which a tumor tissue or the like is not transplanted is used, or alternatively, a urinary cancer model non-human mammal to which a candidate substance is not administered, or the subject animal before administration of a candidate substance can be used as the control animal.

A candidate substance to be administered to the subject animal is not limited, and examples include natural or artificially synthesized various peptides, proteins (including enzymes and antibodies), nucleic acids (such as polynucleotides (DNA and RNA), oligonucleotides (such as siRNA), and peptide nucleic acids (PNA), and low molecular weight or high molecular weight organic compounds.

The administration of a candidate substance can be orally or parenterally performed, which is not restrictive, and in either case, any of known administration methods and administration conditions can be employed. The dose can be also appropriately set in consideration of the type and the condition of the subject animal, and the type and the like of the candidate substance.

(b) Detection Step

In this step, active PKCα is detected in urine (urine sample) of the subject animal to which the candidate substance has been administered. As a method for collecting the urine sample, a known method can be applied. The number of times of detecting active PKCα, the period of the detection and the like are not especially limited, and can be appropriately set in consideration of efficacy and the like of an intended agent for preventing or treating cancer.

Methods for detecting and quantitatively determining PKCα in urine are not limited, and the above-described methods can be appropriately employed.

(c) Evaluation Step

In screening an agent for preventing and/or treating cancer, the detected amount (detection level) of active PKCα in urine is evaluated by comparison between, for example, the subject animal to which the candidate substance has been administered, and the control animal to which the candidate substance has not been administered. It is preferable to select, through this evaluation, whether or not the candidate substance is an intended agent for preventing and/or treating cancer.

Specifically, in urinary cancer model non-human mammals having the amount of active PKCα in urine at the same level, one is used as a subject animal to which the candidate substance is administered, and the other is used as a control animal to which the candidate substance is not administered, and in this case, when the amount of active PKCα in urine of the subject animal is reduced to a level lower than the amount of active PKCα in urine of the control animal, the candidate substance can be selected as the intended agent for treating cancer. On the contrary, when the amount of active PKCα in urine of the subject animal is retained at the same level as or increased to a level higher than the amount of active PKCα in urine of the control animal, the candidate substance cannot be selected as the intended agent for treating cancer. In this case, the subject animal itself before the administration of the candidate substance can be a control animal.

Alternatively, in a case where a urinary cancer model non-human mammal in which a tumor tissue or the like has been surgically resected, or a urinary cancer model non-human mammal in which cancer treatment has been completed by a method except for the surgical resection (such as a medical method) is used as a subject animal to which the candidate substance is administered, and a normal non-human mammal is used as a control animal, when the amount of active PKCα in urine of the subject animal is continuously retained at the same level as the amount of PKCα in urine of the control animal, the candidate substance can be selected as an agent for preventing cancer (specifically, an agent for preventing recurrent cancer). On the contrary, when the amount of active PKCα in urine of the subject animal is increased to a level higher than the amount of PKCα in urine of the control animal, the candidate substance cannot be selected as an intended agent for preventing cancer. In this case, the subject animal itself immediately before the administration of the candidate substance (before the recurrence) can be used as the control animal.

It is noted that the screening of agents for preventing and/or treating cancer based on the amount of active PKCα in urine can be performed on a plurality of types of cancer (preferably all the types of cancer), and hence, if a urinary cancer model non-human mammal of a specific type of cancer is created and used as the urinary cancer model non-human mammal, agents for preventing and/or treating the specific type of cancer can be screened.

[Method for Treating Cancer]

A method for treating cancer of the present embodiment includes: a cancer diagnosis step including detecting active PKCα in urine of an examinee; and a step of administering a therapeutic agent to the examinee when he/she is diagnosed to have cancer.

The cancer diagnosis step may be a step of diagnosing whether or not the examinee has cancer based on an index provided by the method for diagnosing cancer described above. In addition, the cancer diagnosis step may be a step of diagnosing whether or not the examinee has cancer based on the method for evaluating a state of cancer described above.

In the step of administering a therapeutic agent to the examinee when he/she is diagnosed to have cancer, the therapeutic agent to be administered to the examinee can be appropriately selected by those skilled in the art depending on the type of the cancer, and for example, when the cancer is urinary cancer, an anthracycline antitumor antibiotic, mitomycin, and BCG are usually used as a transurethral postoperative recurrence preventive agent, and cisplatin and gemcitabine are usually used as a therapeutic agent in many cases. A therapeutic agent selected by the method for screening agents for preventing and/or treating cancer described above can be used. The administration can be orally or parenterally performed, which is not restrictive, and in either case, any of known administration methods and administration conditions can be employed. The dose can be also appropriately set in consideration of the type of the cancer and the condition of the examinee, and the type and the like of the therapeutic agent.

The method for treating cancer of the present embodiment may further include a step of determining, after administering the therapeutic agent, whether or not treatment with the therapeutic agent is effective by monitoring a detected amount of active PKCα or the phosphorylated substrate peptide by the method for evaluating a state of cancer described above. For example, the method may include determining that the treatment is effective when the detected amount of active PKCα or the phosphorylated substrate peptide is reduced after the administration of the therapeutic agent, and/or determining that the treatment is not effective in a case where the detected amount of active PKCα or the phosphorylated substrate peptide is over a prescribed value and averaged at a high value.

### EXAMPLES

The present invention will now be more specifically described with reference to examples, and it is noted that the present invention is not limited to these examples.

### Example 1 Phosphorylation of Substrate Peptide in Cancer Cell Sample

(Synthesis of Substrate Peptide)

Alphatomega (FKKQGSFAKKK) (SEQ ID NO: 1)) to be used as a substrate peptide was synthesized with an automated peptide synthesizer used in accordance with standard Fmoc chemistry procedures. After treatment with trifluoroacetic acid (TFA), the resultant peptide was purified on an Inertsil ODS-3 column (250×20 mm, 3.5 μm; GL Sciences Inc., Tokyo, Japan) with BioCAD perfusion chromatography system (Ikemoto Scientific Technology Co., Ltd.) and A-B linear gradient at a flow rate of 8 ml/min (eluent A: 0.1% TFA-containing water, eluent B: 0.1% TFA-containing acetonitrile).

This peptide was used in the following experiment as PKCα specific substrate peptide. A molecular weight of this substrate peptide FKKQGSFAKKK-NH₂ is 1338 Da, and is increased by 80 Da to 1418 Da through a phosphorylation reaction with PKCα.

(MALDI-TOF MS Analysis)

An α-cyano-4-hydroxycinnamic acid (CHCA) matrix (10 mg/ml) was prepared in 50% water/acetonitrile and 0.1% TFA. This matrix and a sample were mixed in a ratio of 20:1. The resultant specimen/matrix mixture in a total volume of 1 μl was applied on a sample plate, and the resultant was crystallized by drying. In this example, Auto Flex Speed manufactured by Bruker was used in positive or negative mode. An acceleration voltage was set to 20 kV, and extraction delay time was set to 100 ns. Typically, 100 laser shots were averaged to improve a signal/noise ratio. All spectra were analyzed with Flex Analysis software (Bruker). A phosphorylation percentage was defined as an ionic strength ratio between a phosphorylated substance and a non-phosphorylated substance, and was calculated in accordance with existing description (Kang, J. -H. et al., (2007) J. Am. Soc. Mass Spectrom. 18, 106-112; and Kang J. -H. et al., (2007) J. Am. Soc. Mass Spectrom. 18, 1925-1931).

In order to examine whether or not the PKCα specific substrate peptide could be phosphorylated by bladder cancer, culture cells derived from 5 different bladder cancers (KU-1, KU-7, T24, TCCSUP, and UMUC-3) were analyzed by MALDI-TOF MS. KU-1, KU-7, TCCSUP, and UMUC-3 were maintained in Dulbecco's modified Eagle's media supplemented with 10% fetal bovine serum, penicillin (100 U/ml), streptomycin (100 μg/ml) and amphotericin B (0.25 μg/ml; all Invitrogen). T24 was maintained in RPMI-1640 medium. The resultant cancer cells were placed in a humidified atmosphere containing 5% CO₂ and 95% air. The resultant culture cells were detached, centrifuged at 1,500 rpm for 5 minutes, and the resultant sediment (cells) was used for a phosphorylation reaction with the substrate peptide. The phosphorylation of the substrate peptide was performed in 30 μl of buffer containing 30 μM synthetic peptide (10 mM HEPES (pH 7.5), 10 mM MgCl₂, 100 μM ATP, and 0.2 mg/ml cells). After performing incubation at 37° C. for 60 minutes, the resultant sample was analyzed by MALDI-TOF MS.

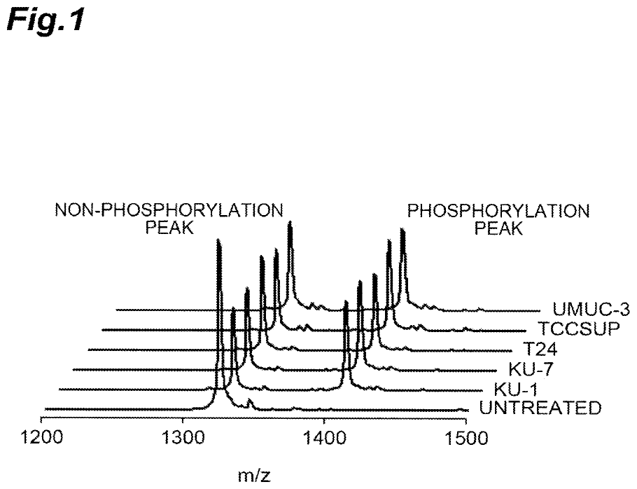

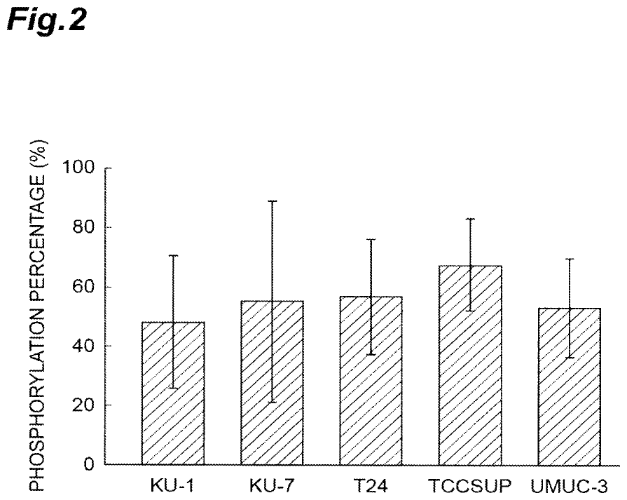

Results are illustrated in FIGS. 1 and 2. “UNTREATED” means containing no cells. The phosphorylation of the substrate peptide was confirmed by the increase of 80 Da in the m/z value, and high phosphorylation percentages were obtained in all samples prepared from bladder cancer-derived culture cells.

### Example 2 Detection of PKCα Activity in Human Urine Sample

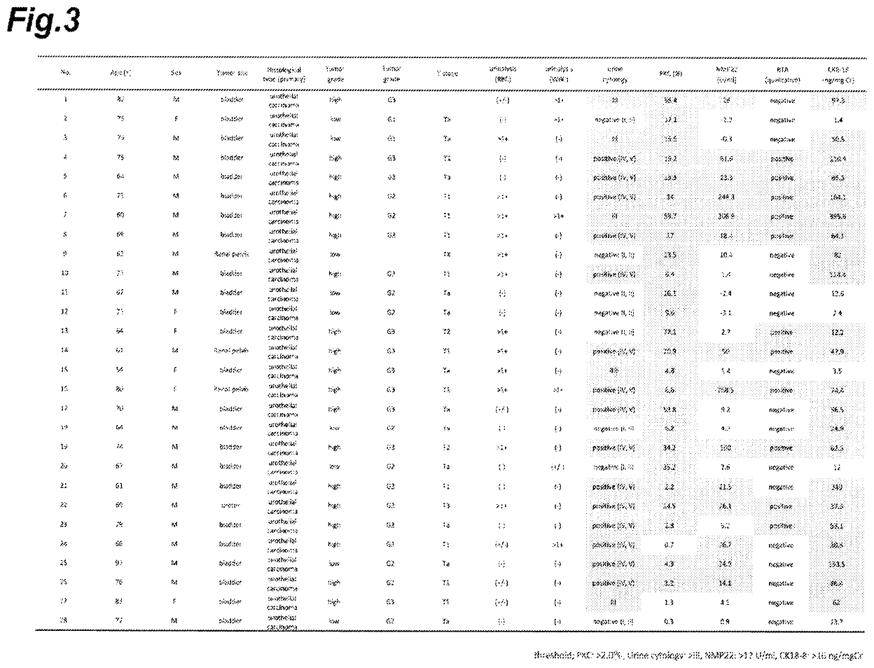

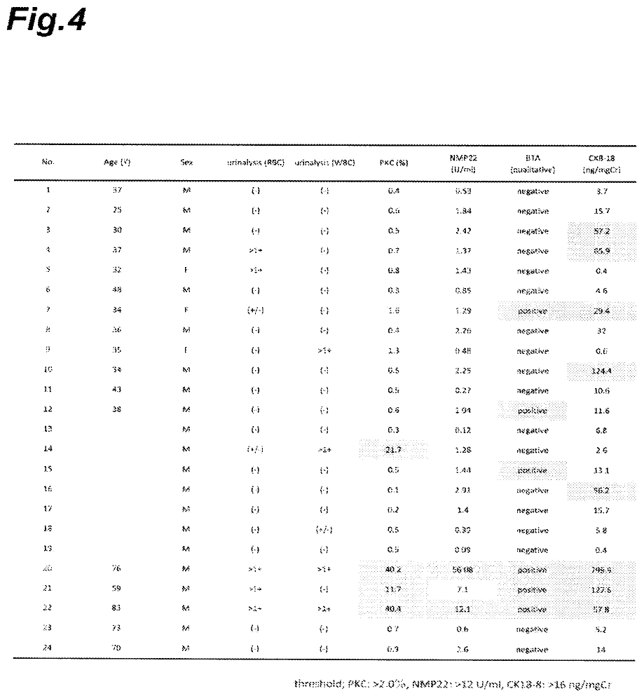

Urines of 28 urothelial carcinoma patients were collected, and used as samples to phosphorylate a substrate peptide. As a control group, urines of 24 non-urothelial carcinoma patients were used. Case information of the urothelial carcinoma patients and the non-urothelial carcinoma patients are illustrated respectively in FIGS. 3 and 4.

20 ml of a urine specimen of each patient was collected, and centrifuged (5000 g, 15 minutes) to collect a sediment. To the resultant, 160 μl of HEPES buffer (containing 10% of sucrose) and 40 μl of complete protease inhibiter cocktail were added for resuspension. The resultant was irradiated with a probe ultrasonic cell disruptor (SONIFIER 250 manufactured by BRANSON) for 10 seconds on ice to disrupt cells. The resultant was centrifuged (5000 g, 15 minutes), a supernatant was collected, and quantitative determination of protein was performed to obtain a protein concentration. The phosphorylation of the substrate peptide was performed with a protein concentration of 0.2 mg/ml, ATP of 100 μM, MgCl₂ of 10 mM, and the substrate peptide of 30 μM. After performing incubation at 37° C. for 60 minutes, the resultant sample was analyzed by MALDI-TOF MS. The analysis of the substrate peptide and by MALDI-TOF MS was performed in the same manner as in Example 1.

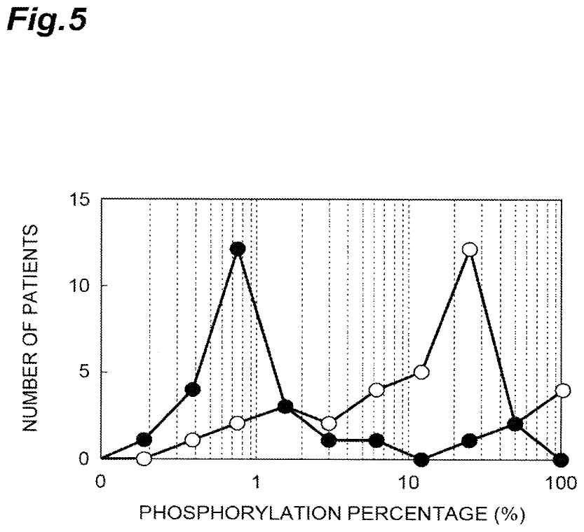

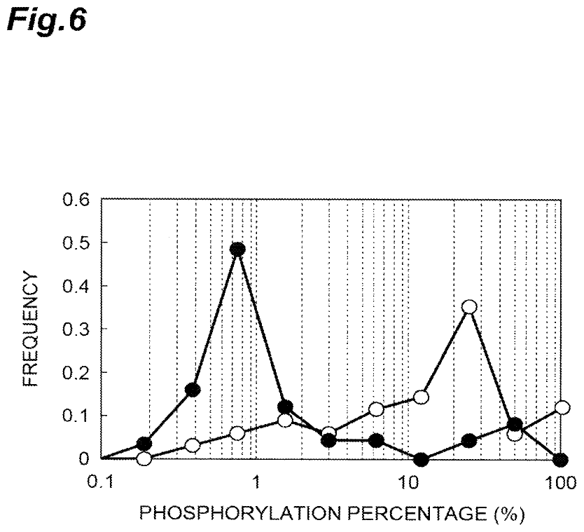

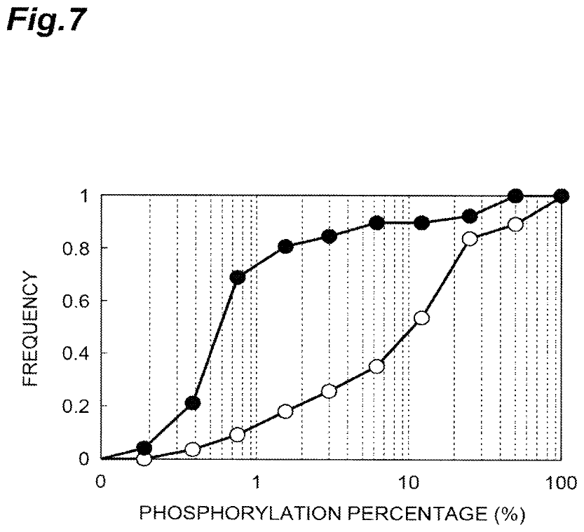

Phosphorylation percentages of the urothelial carcinoma patients and the non-urothelial carcinoma patients were measured to illustrate a histogram (FIG. 5), a probability density function (FIG. 6), and a cumulative distribution function (FIG. 7). The urothelial carcinoma patients exhibited higher phosphorylation as compared with the non-urothelial carcinoma patients, and a phosphorylation percentage of 2% providing a large difference therebetween was set as a threshold.

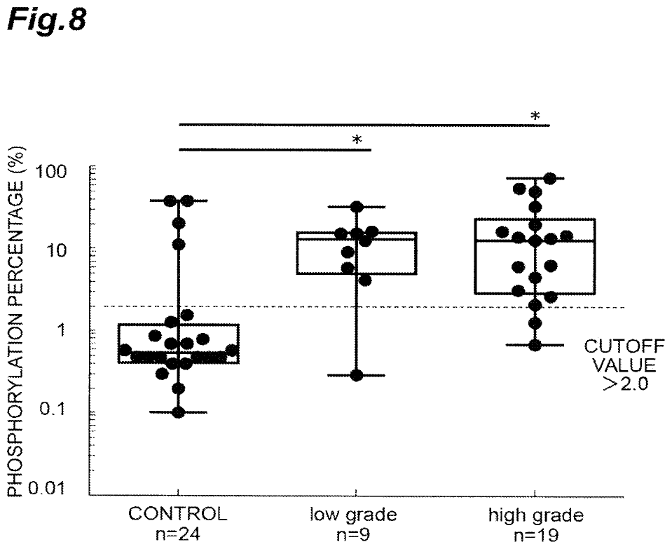

FIG. 8 illustrates the phosphorylation percentages of the substrate peptide in urine samples from 9 low grade urothelial carcinoma patients and 19 high grade urothelial carcinoma patients. Both the low grade and high grade urothelial carcinoma patients exhibited high phosphorylation as compared with the non-urothelial carcinoma patients.

### Example 3 Comparison with Other Urothelial Carcinoma Markers

A threshold of positive reaction was set to 2% based on the phosphorylation percentages of PKCα of the urothelial carcinoma patients and the non-urothelial carcinoma patients calculated in Example 2, and comparison was made with other existing urothelial carcinoma markers (urine cytology, NMP22 (Alere), BTA (Fujirebo Inc.), and CK18-8 (iDL Biotech)). The existing urothelial carcinoma markers were used in accordance with attached documents.

The PKCα threshold of urothelial carcinoma patients was set to 2%, and regarding the other urothelial carcinoma markers, thresholds were set to 16 ng/mgCr for CK18-8, and 12 Um′ for NMP22, and sensitivity, specificity and accuracy of each measurement method were examined. Since urine cytology and BTA are qualitative methods, these were examined by employing positive/negative determination. PKCα exhibited a sensitivity of 89.3%, a specificity of 83.3%, and an accuracy of 86.5%, which were all high. On the other hand, NMP22 exhibited a sensitivity of 50.0%, a specificity of 91.7%, and an accuracy of 69.2%, TBA exhibited a sensitivity of 39.4%, a specificity of 75.0%, and an accuracy of 55.8%, and CK18-8 exhibited a sensitivity of 75.0%, a specificity of 58.3%, and an accuracy of 67.3%, and thus, their specificities were high but sensitivities were low (Table 1).

TABLE 1

| | | | | Specificity | |
|---|---|---|---|---|---|
| | Sensitivity (Positive/Number of Samples) | | | (Negative/Number | |
| | all grades | low grade | high grade | of Samples) | Accuracy |
| PKC | 89.3% (25/28) | 88.9% (8/9) | 89.5% (17/19) | 83.3% (20/24) | 86.5% |
| Urine | 71.4% (20/28) | 22.2% (2/9) | 94.7% (18/19) | — | — |
| Cytology | | | | | |
| NMP22 | 50.0% (14/28) | 11.1% (1/9) | 68.4% (13/19) | 91.7% (22/24) | 69.2% |
| BTA | 39.3% (11/28) | 0.0% (0/9) | 57.9% (11/19) | 75.0% (18/24) | 55.8% |
| CK8-18 | 75.0% (21/28) | 44.4% (4/9) | 89.5% (17/19) | 58.3% (14/24) | 67.3% |

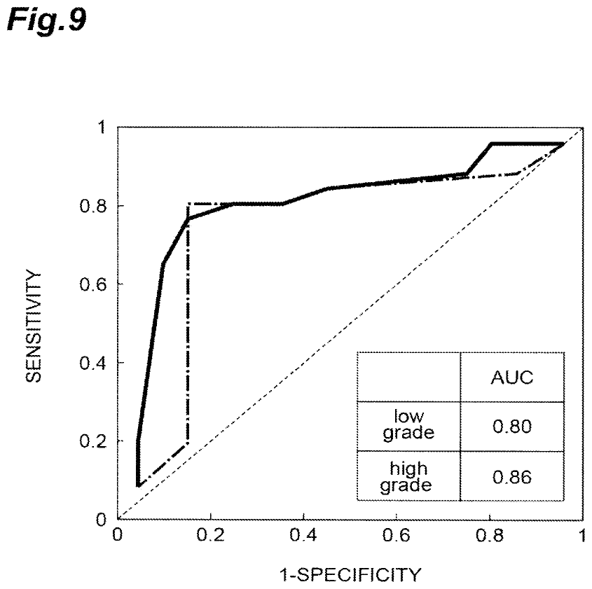

FIG. 9 illustrates receiver operating characteristic (ROC) curves of diagnosis based on the phosphorylation percentage of the substrate peptide using urine samples from 9 low grade urothelial carcinoma patients and 19 high grade urothelial carcinoma patients. Active PKCα in urine exhibited high detection sensitivity and specificity not only in the high grade urothelial carcinoma but also in the low grade urothelial carcinoma.

1. A method for detecting active protein kinase Ca in a subject having or having a risk of cancer comprising:
detecting active protein kinase Ca in urine of the subject.

2. The method according to claim 1, wherein the cancer includes urinary cancer.

3. The method according to claim 1, wherein the cancer includes urothelial carcinoma.

4. The method according to claim 1, wherein the cancer includes carcinoma in situ.

5. (canceled)

6. A cancer diagnosis kit comprising:
an antibody or a substrate peptide for detecting active protein kinase Ca,
wherein diagnosis is made using urine of an examinee.

7. A method for evaluating a state of cancer, comprising
reacting an antibody to the active protein kinase Cα with urine of an examinee and
detecting active protein kinase Ca, or reacting a substrate peptide of active protein kinase Cα with urine of the examinee and detecting the peptide having been phosphorylated,
wherein a state of the cancer is evaluated with an obtained detection result used as an index.

8-9. (canceled)

10. The method according to claim 2, wherein the cancer includes urothelial carcinoma.

11. The method according to claim 2, wherein the cancer includes carcinoma in situ.

12. The method according to claim 3, wherein the cancer includes carcinoma in situ.

13. The kit according to claim 6, wherein the kit further comprises instructions for use of the kit to detect active PKCα in urine of an examinee for cancer diagnosis.

14. The kit according to claim 6, wherein the kit further comprises a urine collecting container.

15. The kit according to claim 13, wherein the kit further comprises a urine collecting container.

16. The method according to claim 1, wherein the subject is diagnosed with cancer.
